(19) United States
(12) Patent Application Publication
(10) Pub. No.: US 2023/0349926 A1
(43) Pub. Date: Nov. 2, 2023

(54) METHODS, COMPOSITIONS, AND KITS FOR DETECTING AND MEASURING ENDOTHELIAL INJURY IN NORMAL AND DISEASED HUMAN CENTRAL NERVOUS SYSTEM (CNS)

(71) Applicant: Ohio State Innovation Foundation, Columbus, OH (US)

(72) Inventor: Rawan TARAWNEH, Columbus, OH (US)

(21) Appl. No.: 18/027,589

(22) PCT Filed: Sep. 22, 2021
(86) PCT No.: PCT/US21/51520

§ 371 Date: Mar. 21, 2023

Related U.S. Application Data
(60) Provisional application No. 63/081,451, filed on Sep. 22, 2020.

(51) Int. Cl. G01N 33/68

(52) U.S. Cl. CPC G01N 33/6896; G01N 2800/2821; G01N 2800/52; G01N 2333/70564; G01N 2333/4722; G01N 2333/7452; G01N 2333/4727

(57) ABSTRACT

Disclosed herein are methods and compositions useful in diagnosing, prognosing, monitoring, and treatment of neurological disorders. The markers are syndecan-1, syndecan-4, thrombomodulin, plasmalemmal vesicle-associated protein, E-selectin, and VE-cadherin. These markers can be used alone or in combination.

### CROSS-REFERENCE TO RELATED APPLICATIONS

This application claims benefit of U.S. Provisional Application No. 63/081,451, filed Sep. 22, 2020, incorporated herein by reference in its entirety.

### BACKGROUND

Endothelial injury is an important pathological mechanism in several neurological disorders, including neurodegenerative, neuroinflammatory, and cerebrovascular disorders, as well as neurological complications of systemic diseases, side effects of immunomodulatory treatments, chemotherapeutic agents, brain radiation, and acute or chronic sequalae of traumatic brain or spinal cord injury.

Endothelial cells are a type of squamous cells which form the inner lining of blood vessels. Endothelial cells play an important role in the brain as they are the main constituent of the blood-brain barrier, which is a highly selective semipermeable border that separates circulating blood from brain tissue and from the extracellular fluid in the brain. Endothelial cells regulate the entry of different types of circulating cells and molecules into the brain and are important in the exchange of nutrients and metabolic waste products between the brain and the systemic circulation. Endothelial cells also regulate the entry of immune cells and inflammatory mediators into the brain, thereby protecting the brain from the deleterious effects of systemic inflammation. Normal endothelium is also important in limiting the entry of cancer cells and infectious particles into the brain. Therefore, intact endothelium protects the brain milieu and is needed for normal brain metabolism, function, and a healthy immune response of the brain to exogenous agents.

Endothelial injury is a common and important mechanism in the pathogenesis of many neurological disorders. These include neuroinflammatory, autoimmune, and demyelinating conditions (e.g. multiple sclerosis, primary and secondary CNS vasculitis, neuromyelitis optica spectrum disorder (NMOSD), Susac syndrome, and sarcoidosis) in which endothelial injury and blood-brain barrier disruption occur as a result of inflammation and contribute to myelin loss and neuronal injury.

Other neurological disorders include cerebrovascular conditions (e.g. hypertensive encephalopathy, encephalopathy in the setting of liver or renal disease, posterior reversible encephalopathy syndrome [PRES], small vessel disease and ischemic or hemorrhagic strokes) in which endothelial injury is a primary mechanism leading to edema and microbleeds. Neurodegenerative disorders (e.g. mild cognitive impairment or dementia due to Alzheimer's disease [AD], Parkinson disease, vascular dementia, Lewy body dementia, and frontotemporal lobar degeneration) are also included. Recent evidence suggests that many neurodegenerative conditions, including Alzheimer disease, involve endothelial injury which promotes and exacerbates the deposition and aggregation of abnormal proteins (e.g. amyloid, tau, and/or synuclein) in the brain tissue. Endothelial injury is an important mechanism in neurological complications of systemic disorders such as liver, pulmonary, and renal disease, connective tissue disorders, rheumatological conditions, and systemic infections (e.g. HIV).

Further included are traumatic brain and spinal cord injury. Endothelial injury is an important consequence of traumatic brain and spinal cord injury including concussions, contusions, and chronic sequalae of brain injury such as chronic traumatic encephalopathy (CTE). Also included are CNS side effects and/or response related to medications or brain radiation. Endothelial injury is considered the main mechanism underlying neurological complications of several chemotherapeutic agents (e.g. cognitive impairment due to chemotherapy referred to as “chemo-brain”), brain or spinal cord radiation (e.g. radiation-induced leukoencephalopathy or radiation-induced vasculitis), and has been shown to modify the brain's response to several therapeutic agents (e.g. monoclonal antibodies, immunosuppressive, and anti-inflammatory treatments). Endothelial injury may influence drug entry into the CNS leading to abnormally higher or lower levels of drugs in the brain. Additionally, endothelial injury may influence drug efficacy by modulating the drug's ability to reach or interact with its therapeutic target in the CNS (e.g. the monoclonal antibody natalizumab interacts with a protein on the endothelial surface [VCAM] to prevent the entry of leukocytes from the circulation into the brain). Together, these findings support the notion that endothelial injury is an important mediator of several neurological disorders, neurological complications of systemic disorders, and neurological complications associated with different treatment modalities.

There are no current biomarkers which can reliably detect or measure endothelial injury in the human CNS in either research or clinical settings. As endothelial injury is an important mechanism in several neurological conditions, and there is a great need to identify novel markers that reflect injury or loss of integrity of the endothelium of the human CNS.

### SUMMARY

The present invention relates to methods of detecting endothelial injury in a subject and treating the subject accordingly, the method comprising detecting elevated levels of at least two of the following markers: syndecan-1, syndecan-4, thrombomodulin, plasmalemmal vesicle-associated protein, E-selectin, and VE-cadherin; and treating the subject accordingly. Specifically, disclosed is detecting at least two, three, four, five, or all six of these markers together.

Further disclosed herein is a method of treating a neurodegenerative disorder in a subject, wherein the method comprises detecting elevated levels of at least two of the following endothelial markers: syndecan-1, syndecan-4, E-selectin, and VE-cadherin; and treating the subject with elevated levels of two or more of these markers with treatments for a neurodegenerative disorder.

Also disclosed herein is a method of treating a subject based on progression of endothelial injury in the subject, the method comprising: measuring levels of at least two of the following endothelial markers in the subject: syndecan-1, syndecan-4, thrombomodulin, plasmalemmal vesicle-associated protein, E-selectin, and VE-cadherin; after a period of time, again measuring levels of at least two of the following endothelial markers in the subject: syndecan-1, syndecan-4, thrombomodulin, plasmalemmal vesicle-associated protein, E-selectin, and VE-cadherin; determining a significant change in the level of the markers; and modifying treatment strategy based on a significant change in the level of markers.

Also disclosed herein is a method of monitoring effects of a composition on endothelial injury in a subject, the method comprising: measuring levels of at least two of the following endothelial markers in the subject: syndecan-1, syndecan-4, thrombomodulin, plasmalemmal vesicle-associated protein, E-selectin, and VE-cadherin; administering the composition to the subject; again measuring levels of at least two of the following endothelial markers in the subject: syndecan-1, syndecan-4, thrombomodulin, plasmalemmal vesicle-associated protein, E-selectin, and VE-cadherin; determining a significant change in the level of the markers; and modifying an amount of the composition given, or discontinuing administration of the composition.

Also disclosed herein are kits. The kit can comprise components for detecting at least two of the following markers: syndecan-1, syndecan-4, thrombomodulin, plasmalemmal vesicle-associated protein, E-selectin, and VE-cadherin.

Further disclosed is a method of treating neuroinflammatory, autoimmune, and demyelinating conditions in a subject, wherein the method comprises detecting elevated levels of at least two of the following markers: syndecan-1, syndecan-4, thrombomodulin, plasmalemmal vesicle-associated protein, E-selectin, and VE-cadherin; and treating the subject with elevated levels by administering steroids to the subject.

Disclosed is a method of treating neuroinflammatory, autoimmune, and demyelinating conditions in a subject, wherein the method comprises detecting elevated levels of at least two of the following markers: syndecan-1, syndecan-4, thrombomodulin, plasmalemmal vesicle-associated protein, E-selectin, and VE-cadherin; and treating the subject with elevated levels by administering aggressive steroid-sparing treatment to the subject.

Also disclosed is a method of detecting neuroinflammatory, autoimmune, and demyelinating conditions in a subject, wherein the method comprises detecting elevated levels of at least two of the following markers: syndecan-1, syndecan-4, thrombomodulin, plasmalemmal vesicle-associated protein, E-selectin, and VE-cadherin; and referring the subject for more frequent monitoring based on presence of elevated levels of markers.

Further disclosed is a method of predicting severity of disease and/or rates of progression of neuroinflammatory, autoimmune, and demyelinating conditions in a subject, wherein the method comprises detecting elevated levels of at least two of the following markers: syndecan-1, syndecan-4, thrombomodulin, plasmalemmal vesicle-associated protein, E-selectin, and VE-cadherin; and referring the subject for more frequent monitoring based on presence of elevated levels of markers.

Disclosed is a method of predicting severity of disease and/or rates of progression of neuroinflammatory, autoimmune, and demyelinating conditions in a subject, wherein the method comprises detecting elevated levels of at least two of the following markers: syndecan-1, syndecan-4, thrombomodulin, plasmalemmal vesicle-associated protein, E-selectin, and VE-cadherin; and treating the subject for the condition based on elevated levels of markers in the subject.

Also disclosed is a method of treating a cerebrovascular condition in a subject, wherein the method comprises detecting elevated levels of at least two of the following markers: syndecan-1, syndecan-4, thrombomodulin, plasmalemmal vesicle-associated protein, E-selectin, and VE-cadherin; and treating the subject with elevated levels with treatments for preventing hemorrhage in the subject.

Further disclosed is a method of treating a cerebrovascular condition in a subject, wherein the method comprises detecting elevated levels of at least two of the following markers: syndecan-1, syndecan-4, thrombomodulin, plasmalemmal vesicle-associated protein, E-selectin, and VE-cadherin; and subsequently monitoring the subject for microbleeds associated with cerebrovascular conditions, wherein said subject diagnosed with a microbleed is treated accordingly.

Disclosed is a method of monitoring and differentially treating a subject undergoing treatment for a cerebrovascular condition, wherein the method comprises detecting elevated levels of at least two of the following markers: syndecan-1, syndecan-4, thrombomodulin, plasmalemmal vesicle-associated protein, E-selectin, and VE-cadherin; and determining whether the subject is responding to the treatment, and adjusting the treatment accordingly.

Also disclosed is a method of detecting a cerebrovascular condition in a subject, wherein the method comprises detecting elevated levels of at least two of the following markers: syndecan-1, syndecan-4, thrombomodulin, plasmalemmal vesicle-associated protein, E-selectin, and VE-cadherin; and referring the subject for more frequent monitoring based on presence of elevated levels of markers.

Further disclosed is a method of predicting severity of disease and/or rates of progression of a cerebrovascular condition in a subject, wherein the method comprises detecting elevated levels of at least two of the following markers: syndecan-1, syndecan-4, thrombomodulin, plasmalemmal vesicle-associated protein, E-selectin, and VE-cadherin; and referring the subject for more frequent monitoring based on presence of elevated levels of markers.

Disclosed is a method of predicting severity of disease and/or rates of progression a of cerebrovascular condition in a subject, wherein the method comprises detecting elevated levels of at least two of the following markers: syndecan-1, syndecan-4, thrombomodulin, plasmalemmal vesicle-associated protein, E-selectin, and VE-cadherin; and treating the subject for the condition based on elevated levels of markers in the subject.

Also disclosed is a method of monitoring and differentially treating a subject undergoing treatment for cerebrovascular conditions, wherein the method comprises detecting elevated levels of at least two of the following markers: syndecan-1, syndecan-4, thrombomodulin, plasmalemmal vesicle-associated protein, E-selectin, and VE-cadherin; and determining whether the subject is responding to the treatment, and adjusting the treatment accordingly.

Further disclosed is a method of detecting a neurodegenerative disorder in a subject, wherein the method comprises detecting elevated levels of at least two of the following markers: syndecan-1, syndecan-4, thrombomodulin, plasmalemmal vesicle-associated protein, E-selectin, and VE-cadherin; and referring the subject for more frequent monitoring based on presence of elevated levels of markers.

Disclosed is a method of predicting severity of disease and/or rates of progression of a neurodegenerative disorder in a subject, wherein the method comprises detecting elevated levels of at least two of the following markers: syndecan-1, syndecan-4, thrombomodulin, plasmalemmal vesicle-associated protein, E-selectin, and VE-cadherin; and referring the subject for more frequent monitoring based on presence of elevated levels of markers.

Also disclosed herein is a method of predicting severity of disease and/or rates of progression of a neurodegenerative disorder in a subject, wherein the method comprises detecting elevated levels of at least two of the following markers: syndecan-1, syndecan-4, thrombomodulin, plasmalemmal vesicle-associated protein, E-selectin, and VE-cadherin; and treating the subject for the condition based on elevated levels of markers in the subject.

Further disclosed herein is a method of treating traumatic brain and/or spinal cord injury in a subject, wherein the method comprises detecting elevated levels of at least two of the following markers: syndecan-1, syndecan-4, thrombomodulin, plasmalemmal vesicle-associated protein, E-selectin, and VE-cadherin; and treating the subject with elevated levels with treatments for a traumatic brain and/or spinal cord injury.

Also disclosed herein is a method of monitoring and differentially treating a subject undergoing treatment for traumatic brain and/or spinal cord injury, wherein the method comprises detecting elevated levels of at least two of the following markers: syndecan-1, syndecan-4, thrombomodulin, plasmalemmal vesicle-associated protein, E-selectin, and VE-cadherin; and determining whether the subject is responding to the treatment, and adjusting the treatment accordingly.

Disclosed herein is a method of detecting traumatic brain and/or spinal cord injury in a subject, wherein the method comprises detecting elevated levels of at least two of the following markers: syndecan-1, syndecan-4, thrombomodulin, plasmalemmal vesicle-associated protein, E-selectin, and VE-cadherin; and referring the subject for more frequent monitoring based on presence of elevated levels of markers.

Further disclosed herein is a method of predicting severity of disease and/or rates of progression of a traumatic brain and/or spinal cord injury in a subject, wherein the method comprises detecting elevated levels of at least two of the following markers: syndecan-1, syndecan-4, thrombomodulin, plasmalemmal vesicle-associated protein, E-selectin, and VE-cadherin; and referring the subject for more frequent monitoring based on presence of elevated levels of markers.

Also disclosed herein is a method of predicting severity of disease and/or rates of progression of a traumatic brain and/or spinal cord injury in a subject, wherein the method comprises detecting elevated levels of at least two of the following markers: syndecan-1, syndecan-4, thrombomodulin, plasmalemmal vesicle-associated protein, E-selectin, and VE-cadherin; and treating the subject for the condition based on elevated levels of markers in the subject.

Disclosed herein is a method of treating a neurological complication of a systemic disorder in a subject, wherein the method comprises detecting elevated levels of at least two of the following markers: syndecan-1, syndecan-4, thrombomodulin, plasmalemmal vesicle-associated protein, E-selectin, and VE-cadherin; and treating the subject with elevated levels with treatments for a neurological complication of a systemic disorder.

Also disclosed is a method of monitoring and differentially treating a subject undergoing chemotherapy treatment, wherein the method comprises detecting elevated levels of at least two of the following markers: syndecan-1, syndecan-4, thrombomodulin, plasmalemmal vesicle-associated protein, E-selectin, and VE-cadherin; and further monitoring the subject to determine whether the subject has cognitive impairment due to chemotherapy.

Further disclosed is a method of detecting neurological complication of a systemic disorder in a subject, wherein the method comprises detecting elevated levels of at least two of the following markers: syndecan-1, syndecan-4, thrombomodulin, plasmalemmal vesicle-associated protein, E-selectin, and VE-cadherin; and referring the subject for more frequent monitoring based on presence of elevated levels of markers.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1A-D shows analyses from a study showing that individuals with Alzheimer Disease (AD) had higher levels of CSF syndecan-1 (14.8 ng/ml; p=0.04) (FIG. 1A), thrombomodulin (13.7 ng/ml; p=0.05) (FIG. 1B), pl-VAP (44.6 ng/ml, p=0.04) (FIG. 1C), and VE-cadherin (25.1 ng/ml; p=0.02) (FIG. 1D) compared to cognitively normal controls (5.1, 5.9, 25.9, and 11.9, respectively).

FIG. 2A-B shows CSF VE-cadherin (FIG. 2A) and E-selectin (FIG. 2B) levels were significantly elevated in AD (11.6 ng/ml; p=0.01, and 17.0 ng/ml; p=0.002, respectively) compared to controls (4.5 ng/ml, and 11.7 ng/ml respectively).

FIG. 3A-B shows plasma syndecan-1 (55.7 ng/ml, p=0.04) (FIG. 3A) and thrombomodulin (9.7 ng/ml, p=0.05) (FIG. 3B) levels were significantly increased in thrombotic thrombocytopenic purpura (TTP) patients who had evidence of cognitive impairment (CI) compared to cognitively normal individuals with TTP (CN; 34.7 ng/ml and 3.5 ng/ml, respectively).

FIG. 4 shows CSF syndecan-1 (SDC1) levels were elevated in individuals with MS/autoimmune disease (8.0 ng/ml; p=0.015) compared to controls (3.7 ng/ml).

FIG. 5A-D shows the diagnostic value of CSF markers of endothelial injury in AD. CSF SDC1 levels were significantly elevated in biomarker-confirmed AD (mean±SE, 103.8±6 pg/ml; n=56) compared to healthy controls (52.8±4.6 pg/ml; n=29) and non-AD dementias (61.6±4 pg/ml, n=12, p<0.0001) (FIG. 5A). CSF VE-cadherin (VEC), syndecan-4 (SDC4), and E-selectin (SELE) levels were examined in a subset of this cohort (n=48). CSF VEC levels were significantly higher in AD (mean±SE; 13.1±0.6 ng/ml; n=24) compared to healthy controls (9.1±0.4 ng/ml; n=12) and non-AD dementias (9.2±0.7 ng/ml; n=12) (p<0.0001) (FIG. 5B). Similarly, CSF SDC4 (61.3±5 pg/ml; n=23) and SELE (57.3±3 pg/ml; n=24) levels were significantly elevated in AD compared to controls (SDC4, 21.5±3 pg/ml; SELE, 28.9±3 pg/ml; n=12) and non-AD dementias (SDC4, 27.4±3 pg/ml; SELE, 32.8±3 pg/ml, n=12) (p<0.0001) (FIG. 5C-D), adjusting for age, gender, and the APOE4 genotype.

FIG. 6A-B shows the prognostic value of CSF SDC1 in predicting clinical progression in AD severity over time.

FIG. 7A-C shows endothelial markers correlate with cognitive outcomes in AD. Higher CSF levels of endothelial markers, reflective of more severe endothelial injury, were associated with lower scores on the Montreal Cognitive Assessment (MOCA) and the Hopkins Verbal Learning Test consistent with more severe cognitive impairment.

FIG. 8A-B shows endothelial markers correlate with synaptic injury in AD.

FIG. 9A-D shows endothelial markers correlate with whole brain and hippocampal atrophy in AD. Higher CSF levels of endothelial markers, reflective of more severe endothelial injury, were associated with lower whole brain (WBV) and hippocampal (HC) volumes, consistent with more severe brain atrophy.

FIG. 10A-C shows that higher CSF levels of syndecan-1 correlate with more severe tau pathology as reflected by higher CSF levels of total tau (t-tau) and tau phosphorylated at threonine 181 (p-tau181) (FIG. 10A-10B). Higher CSF levels of VE-cadherin were associated with lower CSF Aβ42 levels consistent with more severe amyloid pathology. These findings demonstrate that markers of endothelial injury correlate with tau and amyloid pathology in AD (FIG. 10C).

FIG. 11 shows CSF SELE levels correlate with CSF sTREM2 (r=0.45, p=0.02) a surrogate for microglial activation and inflammation. These data suggest that CSF SELE levels reflect CNS endothelial injury which is associated with microglial dysregulation in AD

FIG. 12 shows Western blot analyses of brain-derived exosomes extracted from plasma samples, and plasma samples that did not undergo exosome extraction. This figure shows that SDC1 can be extracted from exosomes derived from the brain endothelium of AD (Clinical Dementia Rating [CDR] 0.5-1) and healthy controls (CDR 0). The data also show that SDC1 levels (estimated by band density on WB) are higher in exosomes derived from the brain endothelium in mild AD dementia (CDR 1; Lane B) and mild cognitive impairment (MCI; CDR 0.5) due to AD (Lanes A and D) compared to brain-derived exosomes from healthy controls (CDR 0; Lane C) and plasma SDC1 levels (Lanes E and F). The molecular weight of SDC-1 is ˜32 k Da; however, the core protein often migrates as an SDS-stable dimer with an approximate molecular weight between 50-70 kDa (arrow)

### DETAILED DESCRIPTION

The following description of the disclosure is provided as an enabling teaching of the disclosure in its best, currently known embodiment. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Definitions

In this specification and in the claims which follow, reference will be made to a number of terms which shall be defined to have the following meanings:

As used herein, the singular forms “a,” “an” and “the” include plural referents unless the context clearly dictates otherwise. Thus, for example, reference to a “metal” includes examples having two or more such “metals” unless the context clearly indicates otherwise.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The term “neurodegenerative diseases” “neurological diseases” “CNS diseases” refers to diseases such as Alzheimer's Disease (AD), as well as diseases such as Creutzfeld-Jakob disease, Parkinson's, senile brain atrophy, Gerstmann-Straussler-Scheinker disease, stroke, PTSD, Tumors, vascular disorders, and host of other such CNS afflictions.

The terms “therapeutic,” “therapeutically effective doses,” and their cognates refer to those doses of a substance that result in prevention or delay of onset, or amelioration, of one or more symptoms of a disease such as Alzheimer's disease.

The terms “therapeutic agents” and “therapy” imply to all drugs used to treat a disease or disorder.

A “subject,” “individual” or “patient” used interchangeably herein, refers to a vertebrate, preferably a mammal, more preferably a human.

The term “mammal (s)” include but are not limited to, humans, mice, rats, monkeys, farm animals, sport animals, and pets.

As used herein the term “ameliorate” is synonymous with “alleviate,” “relief,” or “relieve” and means to reduce or ease signs and symptoms, cure, or curtail the disease processes.

The term “BBB” (blood brain barrier) refers to a network of blood vessels and tissue that is made up of closely spaced cells and helps keep harmful substances from reaching the brain. The blood-brain barrier lets some substances, such as water, oxygen, carbon dioxide, and general anesthetics, pass into the brain. It also keeps out bacteria and other substances, such as many anticancer drugs.

The terms “tumor necrosis factors,” (TNF), or “cytokines” refer to naturally occurring cytokines present in humans or mammals, which plays a key role in the inflammatory immune response and in the response to infection or autoimmune bodies.

A “composition” “compound” or “medicament” encompasses a combination of an active agent or diluents, binder, stabilizer, buffer, salt, lipophilic solvent, preservative, adjuvant or the like, or a mixture of two or more of these substances. Carriers are preferably pharmaceutically acceptable.

The terms “treat,” “treating” and “treatment” “cure” “curtail” used herein, and unless otherwise specified, mean something which reduces, retards, or slows the progression and the severity of the disease using the invention and therapeutic agents described herein.

The term “angiogenesis” refers to a process of tissue vascularization that involves the development of new vessels. Angiogenesis occurs via one of three mechanisms: (1) neovascularization, where endothelial cells migrate out of pre-existing vessels beginning the formation of the new vessels; vasculogenesis, where the vessels arise from precursor cells de novo; or vascular expansion, where existing small vessels enlarge in diameter to form larger vessels (Blood, C. H. and Zetter, 1990, Biochem. Biophys. Acta. 1032:89-118). Angiogenesis is also involved in wound healing and in the pathogenesis of a large number of clinical diseases including tissue inflammation, arthritis, asthma, tumor growth, diabetic retinopathy, and other conditions. Clinical manifestations associated with angiogenesis are referred to as angiogenic diseases (Folkman, J. and Klagsbrun, 1987, Science 235: 442-7).

The term “endothelial cells” means those cells making up the endothelium, the monolayer of simple squamous cells which lines the inner surface of the circulatory system. Endothelial cells also have the capacity to migrate, a process important in angiogenesis. Endothelial cells form new capillaries in vivo when there is a need for them, such as during wound repair or when there is a perceived need for them as in tumor formation. The formation of new vessels is termed angiogenesis, and involves molecules (angiogenic factors) which can be mitogenic or chemoattractant for endothelial cells (Klagsbum, supra). During angiogenesis, endothelial cells can migrate out from an existing capillary to begin the formation of a new vessel the cells of one vessel migrate in a manner which allows for extension of that vessel (Speidel, Am J. Anat. 52: 1-79).

The terms “angiogenic endothelial cells” and “endothelial cells undergoing angiogenesis” and the like are used interchangeably herein to mean endothelial cells (as defined above) undergoing angiogenesis (as defined above). Thus, angiogenic endothelial cells are endothelial cells which are proliferating at a rate far beyond the normal condition of undergoing cell division roughly once a year.

The term “corresponding endothelial cells” “normal or quiescent endothelial cells” and the like are used in order to refer to normal, quiescent endothelial cells contained within the same type of tissue (under normal conditions) when some of the endothelial cells are undergoing angiogenesis and some of the endothelial cells are quiescent

The term “marker” as used herein refers to proteins, polypeptides, glycoproteins, proteoglycans, lipids, lipoproteins, glycolipids, phospholipids, nucleic acids, carbohydrates, etc., small molecules, or other characteristics of one or more subjects to be used as targets for screening test samples obtained from subjects. “Proteins or polypeptides” used as markers in the present invention are contemplated to include any fragments thereof, in particular, immunologically detectable fragments.

The term “protein” refers to a polymeric form of amino acids of any length, i.e. greater than 2 amino acids, greater than about 5 amino acids, greater than about 10 amino acids, greater than about 20 amino acids, greater than about 50 amino acids, greater than about 100 amino acids, greater than about 200 amino acids, greater than about 500 amino acids, greater than about 1000 amino acids, greater than about 2000 amino acids, usually not greater than about 10,000 amino acids, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. Also included by these terms are polypeptides that are post-translationally modified in a cell, e.g., glycosylated, cleaved, secreted, prenylated, carboxylated, phosphorylated, etc, and polypeptides with secondary or tertiary structure, and polypeptides that are strongly bound, e.g., covalently or non-covalently, to other moieties, e.g., other polypeptides, atoms, cofactors, etc.

The term “antibody” is intended to refer to an immunoglobulin or any fragment thereof, including single chain antibodies that are capable of antigen binding and phage display antibodies).

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The term “sample” as used herein relates to a material or mixture of materials containing one or more analytes of interest. In particular embodiments, the sample may be obtained from a biological sample such as cells, tissues, bodily fluids, and stool. Bodily fluids of interest include but are not limited to, amniotic fluid, aqueous humour, vitreous humour, blood (e.g., whole blood, fractionated blood, plasma, serum, exosomes, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate. In particular embodiments, a sample may be obtained from a subject, e.g., a human, and it may be processed prior to use in the subject assay.

The term “analyte” refers to a molecule (e.g., a protein, nucleic acid, or other molecule) that can bound by a capture agent and detected.

The term “assaying” refers to testing a sample to detect the presence and/or abundance of an analyte.

As used herein, the terms “determining,” “measuring,” and “assessing,” and “assaying” are used interchangeably and include both quantitative and qualitative determinations.

The term “capture agent/analyte complex” is a complex that results from the specific binding of a capture agent with an analyte. A capture agent and an analyte for the capture agent will usually specifically bind to each other under “specific binding conditions” or “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens and nucleic acid hybridization are well known in the art (see, e.g., Harlow and Lane (Antibodies: A Laboratory Manual Cold Spring Harbor Laboratory, Cold Spring Harbor, N.Y. (1989) and Ausubel, et al, Short Protocols in Molecular Biology, 5th ed., Wiley & Sons, 2002).

The term “specific binding conditions” as used herein refers to conditions that produce nucleic acid duplexes or protein/protein (e.g., antibody/antigen) complexes that contain pairs of molecules that specifically bind to one another, while, at the same time, disfavor to the formation of complexes between molecules that do not specifically bind to one another. Specific binding conditions are the summation or combination (totality) of both hybridization and wash conditions, and may include a wash and blocking steps, if necessary.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular electrode is disclosed and discussed and a number of modifications that can be made to the electrode are discussed, specifically contemplated is each and every combination and permutation of the electrode and the modifications that are possible unless specifically indicated to the contrary. Thus, if a class of electrodes A, B, and C are disclosed as well as a class of electrodes D, E, and F and an example of a combination electrode, or, for example, a combination electrode comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### GENERAL DESCRIPTION OF THE INVENTION

Using bioinformatics, six proteins which serve as quantitative biomarkers of endothelial injury in the human brain and spinal cord have been identified: syndecan-1; [CD138; UniProtKB-P18827]; syndecan-4 [SDC4; UniProtKB-P31431]; thrombomodulin [CD141; UniProtKB-P07204]; plasmalemmal vesicle-associated protein [pl-VAP; UniProtKB-Q9BX97]; E-selectin [CD62E; UniProtKB-p16581]; and VE-cadherin [UniProtKB-p33151]. These proteins are abundantly expressed in brain endothelium and are released into the extracellular space including the cerebrospinal fluid (CSF) and the systemic circulation in the setting of disease. Blood and/or CSF levels of these proteins, either individually or in combination, directly correlate with the degree and severity of endothelial injury in the CNS.

The ability to accurately measure and quantify blood or CSF levels of endothelial injury markers has significant implications in diagnosis, prognostication, disease monitoring, and therapeutic decision making. Specifically, it will allow clinicians and/or researchers to: 1) Detect the presence, and measure the severity, of endothelial injury in several neurological disorders and/or neurological complications of systemic disease or treatments. This will improve early detection and diagnosis of these conditions and improve the ability to assess disease severity; 2) Monitor disease progression and changes in disease severity during disease course and natural disease progression; 3) Monitor response to treatments that target endothelium by following the direction of change in levels of endothelial injury markers in the course of treatment (e.g. before and after treatment); 4) Detect and measure neurological side effects and neurological complications of systemic disorders or treatments related to endothelial injury.

Specifically, this information has important implications in clinical decision-making, drug choice, and therapeutic assessments as it can guide decision-making by determining the appropriate choice of treatment at an individual level (i.e. risk stratification) based on baseline levels of endothelial injury. Endothelial injury markers can help clinicians and/or researchers identify individuals who are the most likely to benefit from a certain drug (i.e. high likelihood of response) and those who are the most likely to develop side effects (i.e. high-risk patients) based on the presence or absence of CNS endothelial injury at baseline (i.e. prior to treatment). Measuring markers of endothelial injury prior to initiation of treatment provides valuable information which assists clinicians and researchers in determining potential risks and benefits of treatment and deciding the best course of action for each individual.

Based on these findings, disclosed herein is a method for detecting endothelial injury in a subject and treating the subject accordingly, the method comprising detecting elevated levels of at least two of the following endothelial markers: syndecan-1, syndecan-4, thrombomodulin, plasmalemmal vesicle-associated protein, E-selectin, and VE-cadherin; and treating the subject accordingly. Specifically, disclosed is detecting at least two, three, four, five, or all six of these markers together. It is noted that these markers are brain-derived markers, in other words, they originate in the brain. Examples are provided below of how they may be detected in the blood.

Additional biomarker levels can also be measured. Example of additional biomarkers include, but are not limited to, t-tau, p-tau181, p-tau217, and p-tau231 and Aβ42 to detect Alzheimer's Disease (AD). Other examples include vascular cell adhesion molecule [VCAM]-1, intercellular adhesion molecule [ICAM]-1, and endothelial leucocyte adhesion molecule [ELAM]-1, although measures of these vascular markers in AD and other neurodegenerative disorders have been inconsistent and of limited value. Other biomarkers are known to those of skill in the art.

Further disclosed herein is a method of treating a neurodegenerative disorder in a subject, wherein the method comprises detecting elevated levels of at least two of the following endothelial markers: syndecan-1, syndecan-4, E-selectin, and VE-cadherin; thrombomodulin, pl-VAP and treating the subject with elevated levels of two or more of these markers with treatments for a neurodegenerative disorder. In other words, if two or more of the markers are detected, the subject can be treated for neurodegenerative disorder.

These markers can be measured in conjunction with other markers, and other routine and standard tests and measurements can be done to further confirm that the subject has a neurodegenerative disorder. The subject can be treated differently based on the results of the presence of at least two of the markers compared to a subject in which the markers were not detected. The elevated levels can indicate, for example, that the subject has Alzheimer's disease. The subject may also be diagnosed with mild cognitive impairment or dementia due to Alzheimer's disease [AD]. It is also possible that the subject has no signs or symptoms or AD at the time that the markers are measured. Furthermore, the markers can be elevated in subjects with Alzheimer's disease but not with those with Parkinson's disease or Frontotemporal dementia and therefore can improve diagnostic accuracy of AD compared to other dementia types. The endothelial markers can also be used to predict clinical progression in Alzheimer's disease over time. Furthermore, future cognitive impairment in cognitively normal subjects can be predicted.

By “elevated levels” is meant an increase of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% or more compared with a “normal,” or standard control of subjects without endothelial injury. Also contemplated is an increase of 2, 3, 4, 5, 6, 7, 8, 9, or 10-fold or more. It can also be compared to a different time point form the same subject.

By “decreased levels” is meant a decrease of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% or more compared with a “normal,” or standard control of subjects with endothelial injury. Also contemplated is a decrease of 2, 3, 4, 5, 6, 7, 8, 9, or 10 fold or more. It can also be compared to a different time point form the same subject.

The markers can be detected in a number of ways. For example, the markers can be detected in cerebrospinal fluid (CSF). Methods for extracting and testing markers in CSF are known to those of skill in the art. The markers disclosed herein have been found in brain-derived exosomes from the blood so it is possible to measure brain endothelial injury using blood. The method for detecting markers in exosomes can be found in U.S. Pat. No. 9,989,539, herein incorporated by reference in its entirety.

Also disclosed herein is a method of treating a subject based on progression of endothelial injury in the subject, the method comprising: measuring levels of at least two of the following endothelial markers in the subject: syndecan-1, syndecan-4, thrombomodulin, plasmalemmal vesicle-associated protein, E-selectin, and VE-cadherin; after a period of time, again measuring levels of at least two of the following endothelial markers in the subject: syndecan-1, syndecan-4, thrombomodulin, plasmalemmal vesicle-associated protein, E-selectin, and VE-cadherin; determining a significant change in the level of the markers; and modifying treatment strategy based on a significant change in the level of markers.

Also disclosed herein is a method of monitoring effects of a composition on endothelial injury in a subject, the method comprising: measuring levels of at least two of the following endothelial markers in the subject: syndecan-1, syndecan-4, thrombomodulin, plasmalemmal vesicle-associated protein, E-selectin, and VE-cadherin; administering the composition to the subject; again measuring levels of at least two of the following endothelial markers in the subject: syndecan-1, syndecan-4, thrombomodulin, plasmalemmal vesicle-associated protein, E-selectin, and VE-cadherin; determining a significant change in the level of the markers; and modifying an amount of the composition given, or discontinuing administration of the composition.

With these methods, a first, or baseline, measurement can be taken, and another measurement can be taken after a given amount of time. For example, the first measurement can be taken when the subject is first diagnosed with the disease or disorder, or it can be done preemptively based on family history or a genetic test which indicates that the subject might be at risk for the disease or disorder. The first measurement can also be done at the initiation, or immediately before, commencement of treatment of a composition begins. The first measurement can also be made at any time point during treatment with a composition.

The second measurement can be made at any time after the first measurement. For example, the second measurement can be made 1, 2, 3, 4, 5, 6, 7, 8, 9, 10, 11, or 12 weeks after the first measurement, or 4, 5, 6, 7, 8, 9, 10, 11, or 12 months after the first measurement, or 2, 3, 4, 5, 6, 7, 8, 9, or 10 years after the first measurement, or any amount in between or shorter or longer than these time periods.

By “significant change” is meant a change which is deemed to be statistically significant. One of skill in the art can readily identify what this means, but for example can mean a change of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% or more compared with a standard control. Also contemplated is a change of 2, 3, 4, 5, 6, 7, 8, 9, or 10 fold or more.

The amount of the composition can be decreased or discontinued when a significant increase in at least one of the endothelial markers is found. By “decreased” is meant reduced by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99%. By “discontinued” is meant stopped entirely.

The amount of the composition can be increased when a significant increase in at least one of the endothelial markers is found. By “increase” is meant increased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or 2, 3, 4, 5, 6, 7, 8, 9, or 10 fold or more.

The composition being monitored can be one that is being used to treat a disease caused by or related to endothelial injury. Examples of diseases and disorders related to endothelial injury, as well as compositions for the treatment of such diseases and disorders, can be found in U.S. Pat. No. 6,930,099, herein incorporated by reference in its entirety for its teaching concerning endothelial injury and dysfunction and treatment options based thereon. When the composition being given is used to treat endothelial injury, one would expect that the markers of endothelial injury would be decreased. However, if such a decrease is not found, the dose, route of administration, or frequency of administration of the composition could be changed. For example, the dose could be increased as described above. If the frequency is changed, it could be that the subject requires a more frequent administration of the composition of the same dose, or a more frequent administration of a higher dose.

Also contemplated is that composition being monitored can be one that is being used to treat a disease which is not caused by or related to endothelial injury. In this case, it could be that the composition is being monitored to assess whether it is causing endothelial injury or not in the subject. For example, the FDA-approved treatment for Alzheimer's Disease, aducanumab, has a significant risk of side effects which are due to endothelial injury. These are referred to as amyloid related imaging abnormalities (ARIAs) and can be in the form of edema (ARIA-E) or hemorrhage (ARIA-H): both of which are due to the antibody damaging the endothelium. The markers disclosed herein can improve the ability to predict, track, and monitor endothelial injury to a much better extent than imaging alone. Importantly, it can be used to stratify patients based on their risk for ARIA before treatment, so that those with higher baseline endothelial injury markers could be classified as being at a higher risk for ARIAs with treatment.

By way of further example, several chemotherapeutic agents, including alkylating (e.g. cisplatin), and anti-angiogenesis agents which block vascular endothelial growth factor (e.g. bevacizumab, sunitinib, and sorafenib), are associated with systemic (e.g. hypertension and myocardial ischemia) and neurological (e.g. cerebral microbleeds and PRES) complications attributed to endothelial dysfunction. Measuring levels of endothelial markers prior to treatment can assist clinicians in determining whether these agents are an appropriate therapeutic choice in a certain individual. For example, high baseline levels of endothelial injury markers in a patient with recurrent glioblastoma multiforme (brain tumor) can alert the clinician that the use of bevacizumab in this patient may be associated with increased CNS toxicity and a higher risk of microbleeds as this patient already has significant endothelial damage that could be exacerbated with the use of this medication. In this case, the clinician may decide to choose an alternative chemotherapeutic agent for treatment.

The results of the assays disclosed herein can also guide decision-making by monitoring therapeutic response to drugs. Measuring endothelial injury markers in individuals who are being treated for a neurological disorder which is associated with endothelial injury is useful in determining whether or not these individuals are responding appropriately to the provided treatments. This information will allow clinicians and/or researchers to modify the choice or dosage of drug accordingly in order to ensure a better response.

For example, CNS vasculitis, which could be primary (i.e. idiopathic) or secondary to connective tissue disorders (e.g. systemic lupus erythematosus [SLE]), drug hypersensitivity reactions, or post-viral syndrome (e.g. Susac disease) is associated with endothelial injury as a result of inflammation of the blood vessel wall. These disorders are usually treated with steroids and/or steroid-sparing immunosuppressive treatments (e.g. intravenous immunoglobulin, methotrexate, and cyclophosphamide). The endothelial markers proposed herein are useful to determine whether individuals with these disorders are responding to treatment (e.g. demonstrating reduced levels of these markers post-treatment compared to pre-treatment levels can indicate response to treatment, while levels that continue to rise despite treatment suggest a poor response to that drug). Changes in levels of endothelial markers can precede clinical evidence of response and therefore allow clinicians to modify the course of treatment early on in the course as these treatments often have a delayed clinical response. Furthermore, these markers can allow assessment of response to treatment in individuals in whom clinical exams may be inaccurate or not possible (e.g. comatose and unresponsive patients).

By way of another non-limiting example, PRES (posterior reversible encephalopathy syndrome) is a serious, potentially life-threatening condition, which is associated with headaches, visual changes, and seizures due to disruption of the blood-brain barrier and endothelial injury predominantly affecting the posterior part of the brain. It is frequently seen in the setting of uncontrolled hypertension, renal failure, or as a side effect of certain medications. Treatment of PRES is based on treating the underlying cause and maintaining normal blood pressure. However, symptomatic improvement is often delayed by weeks or months following treatment. Following levels of endothelial injury markers in patients being treated for PRES facilitates early assessment of a patient's response to therapy prior to change in clinical signs or symptoms and allows clinicians to modify the course of treatment early in the disease course and prevent deleterious consequences such as blindness or brain edema.

The result of the assays disclosed herein can also inform decision-making by determining whether an individual who is being treated for a neurological disorder has developed endothelial injury as a side effect of treatment (e.g. medication or radiation). This information can be used by clinicians and/or researchers to determine whether this treatment modality should be adjusted, and potentially reduced or discontinued.

By way of another non-limiting example, following CSF levels of endothelial injury markers in a cancer patient being treated with cisplatin or bevacizumab can allow the clinician to determine whether this patient has early signs of endothelial dysfunction due to these agents. If CSF endothelial markers were found to be significantly increased above normal, the clinician may decide to discontinue the treatment or modify the dose to avoid severe or permanent neurological complications such as intracranial bleeds due to treatment.

In yet another non-limiting example, anti-amyloid antibodies used in clinical trials of Alzheimer's disease have been associated with increased risk for endothelial damage due to the antibodies binding to amyloid deposited in the brain vessels (referred to as amyloid-related imaging abnormalities [ARIAS]). This immune complex reaction that targets the endothelium can result in serious complications such as intracranial hemorrhage and life-threatening brain swelling. The use of endothelial injury markers can facilitate the early detection of this side effect, and allow researchers to stop these treatments, before intracranial hemorrhage and edema develop.

The results of the assays disclosed herein can also determine the future course of disease, including severity and rate of progression over time, and improve the accuracy of prognostic assessments. In certain conditions, these markers can also be used to assess the likelihood of response to treatment based on baseline levels of these biomarkers. For example, patients with CNS vasculitis who have higher levels of these markers are likely to have more aggressive disease, more rapid rates of disease progression, and poorer outcomes compared to those with lower baseline levels of endothelial markers.

The assays disclosed herein can also be used to detect early signs of neurological injury in the setting of systemic disorders prior to the onset of clinical signs or symptoms. For example, brain radiation is used to treat different types of brain tumors. Radiation can cause damage to the endothelium and the white matter tracts, the clinical manifestations of which are often delayed by several months to years following treatment. Endothelial markers can allow the early detection of radiation induced vasculitis and the initiation of treatment (with steroids or other immunomodulatory treatments) prior to its progression to irreversible neuronal damage and brain atrophy. Early detection in this case is likely to be associated with improved outcomes.

In yet another non-limiting example, patients with TTP have been shown to develop a severe cognitive and behavioral syndrome at a young age. This has been attributed to “subclinical” endothelial damage that progresses over time independent of their clinical relapses of TTP. It has been suggested that treating patients between TTP relapses can help in treating this condition. The use of blood markers of endothelial injury in TTP patients can assist in identifying the subset who are likely to develop this cognitive and behavioral syndrome, and therefore these patients may benefit from higher intensity, or higher frequency, of TTP treatments prior to the progression of the cognitive decline.

The results of the assays disclosed herein can also assist in the evaluation and differential diagnosis of neurological disorders. This information is important in determining the course of treatment, choice, and timing of medication. By way of non-limiting example, a patient with uncontrolled hypertension due to advanced renal failure, who also has pneumonia and a urine infection, is being evaluated for altered mental status (AMS). Each of these 3 factors can cause AMS; however, the clinician is not able to determine with certainty which one of these 3 factors is the main cause of encephalopathy in this patient. The detection of high levels of endothelial injury markers in the CSF or blood of this patient suggests that his encephalopathy is due to the high blood pressure (and renal failure) since pneumonia and UTI are not typically associated with endothelial injury in the brain and cause AMS via different mechanisms.

The endothelial injury markers disclosed herein can also be used in basic and translational research, including animal models, to better understand the role of endothelial damage in the pathogenesis of several disorders and its interactions with other pathologies. This technology improves the understanding of several neurological diseases from a pathophysiological and molecular standpoint and potentially shed light on novel targets for drug discovery.

The findings disclosed herein support the utility of CSF syndecan-1 (CD138), syndecan-4 (SDC4), thrombomodulin (CD141), plasmalemmal vesicle-associated protein (pl-VAP), VE-cadherin, and E-selectin (CD62E) (individually or in combination) as novel markers of endothelial injury in neurological disorders and/or neurological complications of systemic diseases or treatments. By “individually or in combination” is meant that the six markers can each be used individually, or can be used in any combination or permutation possible.

The endothelial markers disclosed herein can be used to detect and treat neuroinflammatory, autoimmune, and demyelinating conditions (e.g. multiple sclerosis, primary and secondary CNS vasculitis, neuromyelitis optica spectrum disorder (NMOSD), Susac syndrome, autoimmune and paraneoplastic encephalitis, and sarcoidosis), for example. The result of the assays using the markers disclosed herein can be used in informing clinical decision making by deciding course of treatment whether patients require treatment with steroids alone or would require initiation of more aggressive steroid-sparing treatment based on baseline levels of these markers in primary or secondary vasculitis, NMOSD, Susac syndrome and sarcoidosis (i.e. higher baseline levels of endothelial injury markers would inform the need for more aggressive treatments).

Following the levels of endothelial markers can assist in monitoring and assessing response to treatment in cases of vasculitis, NMOSD, Susac syndrome, and sarcoidosis (i.e. treatment response being suggested by a reduction in these marker levels after compared to before treatment). Measuring levels of endothelial markers in multiple sclerosis can assist in choice of treatments as determining whether some immunomodulatory treatments such as natalizumab may be appropriate (i.e. high levels of endothelial markers may preclude the use of this medication or suggest lower efficacy of this drug or higher toxicity associated with its use). The markers can also be useful in predicting the severity of disease and rates of progression (i.e. higher baseline levels are indicative of worse outcomes). Early detection of these disorders in individuals with no or mild symptoms allows for more frequent monitoring.

The endothelial markers disclosed herein can be used to detect and treat cerebrovascular conditions (e.g. hypertensive encephalopathy, encephalopathy in the setting of liver or renal disease, posterior reversible encephalopathy syndrome [PRES], small vessel disease and ischemic or hemorrhagic strokes).

Following the levels of endothelial markers can assist in monitoring and assessing response to treatment, such as by demonstrating lower levels of these markers after compared to before treatment. This is particularly important since clinical response to treatment of these conditions is often delayed by days to weeks. The use of these markers allows clinicians to assess the response to treatment at an earlier stage and therefore modify treatment plans early in the disease course to improve outcomes and prevent complications. It also allows for monitoring disease progression over time, and predicting disease course and severity.

Detecting the levels of endothelial markers can also assist in assessing response to risk of hemorrhage and/or identifying early microbleeds associated with these conditions which can be used to modify the dose of other medications often used in the treatment of these conditions which may be associated with bleeding (e.g. antiplatelets and anticoagulants). —Early detection of these disorders in individuals with no or mild symptoms can indicate that more frequent monitoring is needed.

Following the levels of endothelial markers can assist in predicting disease severity and rates of progression, and monitoring disease progression over time. Most of the investigational disease-modifying treatments for AD involve the use of monoclonal antibodies directed against different forms of the amyloid protein. As this protein also deposits in brain blood vessels, the use of these agents has been complicated by vascular injury leading to a higher risk of intracranial bleeding and brain swelling (i.e. edema). The use of endothelial injury markers allow for the detection of the earliest signs of endothelial injury (which occurs as a result of vascular injury) which allows clinicians to reduce the dose or stop these treatments prior to the occurrence of clinically significant complications and measure levels of these markers to decide whether further actions are needed.

The use of these markers in research provides valuable insight regarding the presence and significance of endothelial injury as pathogenic disease mechanisms which provides novel drug targets for the treatment of these conditions and provide useful outcome measures in clinical trials. —Early detection of these disorders in individuals with no or mild symptoms indicates that frequent monitoring should be performed.

The endothelial markers disclosed herein can be used to detect and treat traumatic brain and spinal cord injury (mild, moderate or severe), single or recurrent, including concussions, contusions, diffuse axonal injury (DAI), and chronic traumatic encephalopathy (CTE).

Following the levels of endothelial markers can assist in identification of endothelial injury in these settings would indicate the need for more frequent and intensive monitoring as individuals with evidence of endothelial injury are at higher risk for life-threatening complications such as intracranial bleeds and brain or spinal cord swelling.

Endothelial damage is considered the primary mechanism underlying chronic traumatic encephalopathy (CTE). As there are currently no in vivo markers for CNS endothelial injury, the use of these markers provide researchers with a way to monitor this disease mechanism and its response to investigational treatments in clinical trials independently of changes in clinical exam. It also can allow for predicting and monitoring disease course and progression.

The endothelial markers disclosed herein can be used to detect and treat neurological complications of systemic disorders (cancer, rheumatological, hematological disorders, liver, renal or pulmonary disease, systemic infections such as HIV), systemic or CNS-specific treatments (chemotherapy, brain or spinal cord radiation, immunomodulatory treatments including monoclonal antibodies).

The use of these markers allows for the early identification of neurological complications associated with rheumatological conditions (systemic lupus erythematosus [SLE], rheumatoid arthritis, Sjogren, mixed connective tissue disease, systemic vasculitides, etc.) such as brain vessel inflammation (i.e. vasculitis) associated with these systemic conditions, which will determine the need for more aggressive immunosuppressive treatment with treatment such as prednisone, rituximab, methotrexate, or cyclophosphamide. Also, it allows for monitoring the course of the neurological involvement in these conditions and/or their response to treatment and predicting rates of progression over time.

These markers can help determine an individual's risk for cognitive impairment due to chemotherapy (e.g. methotrexate, lenalidomide, 5-fluorouracil) which can influence the decision to start these medications. Furthermore, they allow close monitoring of changes in the brain endothelium associated with cognitive changes and can be used to assist in decisions regarding adjusting the choice or dose of these medications. The use of these markers also assist in the diagnosis of the cause of cognitive impairment and whether the cognitive impairment is due to chemotherapy or other factors (e.g. sleep disturbance or mood disorder) in individuals as high levels would suggest chemotherapy as the cause of cognitive decline.

In TTP, the use of these markers can allow clinicians whether patients are at higher risk for cognitive impairment due to this condition which will translate into more frequent monitoring and more frequent treatment with plasma exchange therapy (PLEX) or intravenous immunoglobulin (IVIG) in between clinical relapses.

The use of these markers in patients treated with brain or spinal cord radiation can allow clinicians to detect the earliest signs of side effects such as vasculitis which are often associated with strokes and cognitive decline, and therefore to adjust dose or frequency of treatment to reduce these negative side effects.

In addition to measuring protein levels of these markers in blood and cerebrospinal fluid, protein levels can be measured in urine, saliva, and tears and tissue homogenates obtained from humans or animal studies using ELISAs (enzyme-linked immunoassays) and chemiluminescence assays. mRNA expression (nucleic acid) levels can also be monitored in blood and body fluids. Western blots, immunofluorescence, and immunostaining can be used to identify patterns of regional distribution of these markers in blood vessels of different brain regions. Ligands that can bind to these proteins and positron emission tomography (PET scans) can be used to identify patterns of in vivo binding in the human brain.

Examples of the antibodies used with the disclosed methods and assays include, but are not limited to:
E-Selectin (CD62E) human-specific monoclonal antibody from R&D (Luminex LKT007) modified to allow measurement of CSF levels;
thrombomodulin CD141; Abcam antibody ab46508
VE-cadherin (vascular endothelial cadherin) from R&D (DCADVO) modified to allow measurement of CSF levels;
syndecan-1 (CD138) monoclonal antibody specific for human syndecan-1 from R&D (DY2780); modified to allow measurement of CSF levels;
syndecan-4 monoclonal antibody specific for human syndecan-1 from R&D (DY2918); modified to allow measurement of CSF levels;
pl-VAP; monoclonal antibody specific to human pl-VAP from R&D or MyBioSource, MBS2509189.

Also disclosed herein are kits. The kit can comprise components for detecting at least two of the following markers: syndecan-1, syndecan-4, thrombomodulin, plasmalemmal vesicle-associated protein, E-selectin, and VE-cadherin. In one example, the component used for detection is an antibody. The kit can comprise two, three, four, five, or all six markers, in any combination. The kit can also comprise a capture antibody to detect binding of the first antibody to the marker(s) of interest. The capture antibody can also incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes or reagents for detecting the analyte. The antibodies, calibrators and/or controls can be provided in separate containers or pre-dispensed into an appropriate assay format, for example, into microtiter plates.

Optionally, the kit can include quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

The endothelial markers disclosed herein can be used with proximity ligation assays (PLAs). The necessary reagents and components to detect the endothelial markers can be provided in the form of a kit, for example. PLA combines the analyte specificity of high-affinity antibody-antigen binding with the signal detection and amplification capabilities of real-time polymerase chain reaction (PCR) to achieve a simple yet powerful next-generation protein quantitation platform and therefore, can provide highly accurate and efficient assessments of protein levels in small sample volumes in less than 2 hours. Immunoassays based on the PLA technology utilize a matched pair of target-specific antibodies, each conjugated to a DNA oligonucleotide. During antibody-analyte binding, the two DNA oligos are brought into close proximity, which allows for ligation of the two strands and subsequent creation of a template strand for amplification. Several commercial kits such as the ProQuantum kits from ThermoFisher utilize the PLA technology.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

Several studies have been performed to examine the utility of blood and CSF levels of syndecan-1, syndecan-4, thrombomodulin, pl-VAP, E-selectin, and VE-cadherin in measuring endothelial injury in the human CNS. These studies included cohorts of individuals with different neurological conditions who were enrolled through clinical studies at the Department of Neurology at Ohio State University. Together, these findings support the utility of blood or CSF syndecan-1, syndecan-4 thrombomodulin, pl-VAP, VE-cadherin, and E-selectin as novel measures of endothelial injury in several neurological disorders.

### Example 1: Alzheimer's Disease Study 1

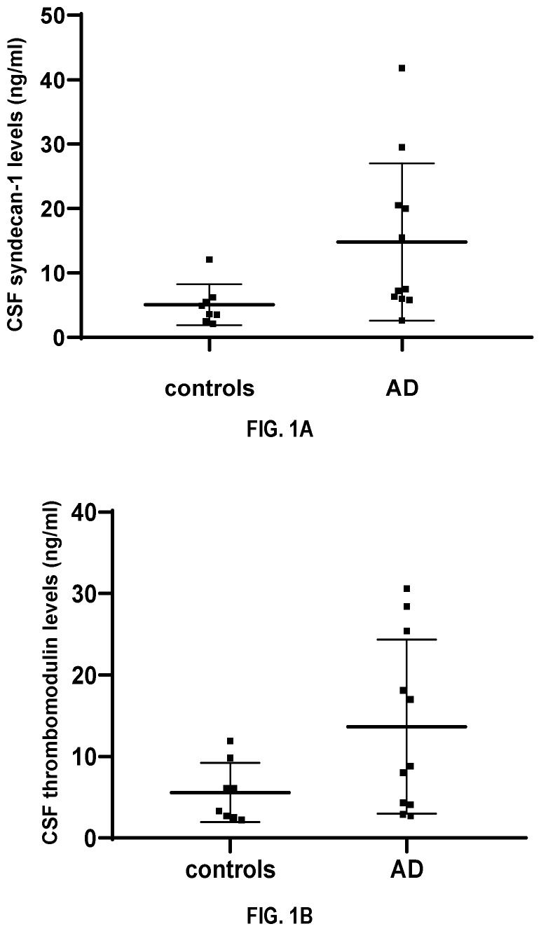

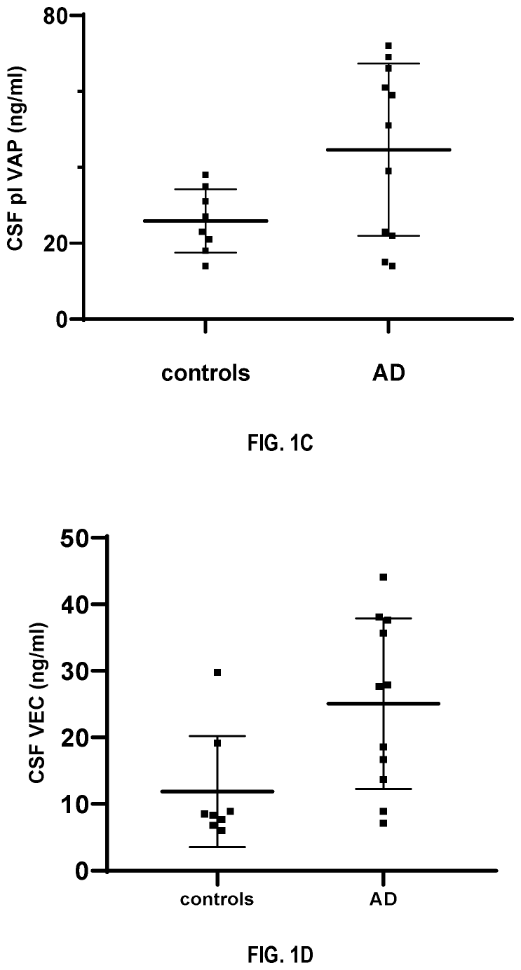

The first study included a cohort of n=11 individuals with a clinical diagnosis of moderate Alzheimer's disease and n=8 cognitively normal controls from the OSU Buckeye Biofluid Repository. Immunoassays based on modifications to commercially available tools were developed in order to allow the quantification of syndecan-1, thrombomodulin, pl-VAP, and VE-cadherin in CSF samples. Analyses from the first pilot study showed that individuals with AD had higher levels of CSF syndecan-1 (14.8 ng/ml; p=0.04), thrombomodulin (13.7 ng/ml; p=0.05), pl-VAP (44.6 ng/ml, p=0.04), and VE-cadherin (25.1 ng/ml; p=0.02) compared to cognitively normal controls (5.1, 5.9, 25.9, and 11.9, respectively) (FIG. 1A-D). These findings show that CSF levels of these markers can measure endothelial injury in the brains of patients with Alzheimer's disease.

### Example 2: Alzheimer's Disease Study 2

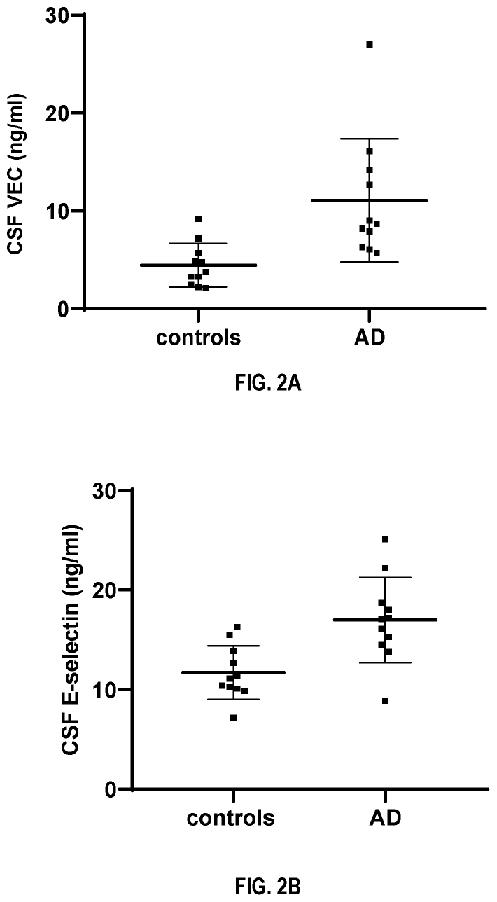

CSF levels of VE-cadherin and E-selectin were measured in an independent cohort of CSF samples including individuals with mild, moderate, or severe AD (n=11) and a group of cognitively normal controls (n=11) using stored CSF samples. In this cohort, CSF VE-cadherin levels and E-selectin were significantly elevated in AD (11.6 ng/ml; p=0.01, and 17.0 ng/ml; p=0.002, respectively) compared to controls (4.5 ng/ml, and 11.7 ng/ml respectively) (FIG. 2). These findings further support the value of these proteins in measuring endothelial injury in Alzheimer's disease.

### Example 3: Hematological Disorders

Immune-mediated thrombotic thrombocytopenic purpura (iTTP) is defined by thrombocytopenia and microangiopathic hemolytic anemia without an alternative explanation, confirmed by severely deficient ADAMTS13 to <10%. It is caused by autoantibodies against the ADAMTS13 protease. Following recovery from an acute iTTP episode, the patient is at risk for relapses and multiple long-term complications including hypertension, depression, headaches and neurocognitive impairment. The etiology of these complications is not well understood. The aim of this study was to identify biomarkers of endothelial injury that could help to diagnose or predict the development of these long-term complications of iTTP. Four plasma biomarkers of endothelial injury were measured in blood samples in patients with TTP with and without cognitive impairment compared to controls.

Syndecan-1 (CD-138), is a cell surface heparan sulfate proteoglycan that interacts with extracellular matrix molecules and growth factors to maintain epithelial cell morphology. It has been reported to be a negative regulator of various inflammatory processes, with Syndecan-1 knockout (Sdc-1−/−) mice showing enhanced disease severity and impaired recovery.

Thrombomodulin (CD141), is an endothelial surface transmembrane glycoprotein. It is involved in the activation of protein C in the inactivation of thrombin. Its expression has been associated with aging and cardiovascular disease.

Plasmalemmal Vascular adhesion protein-1 (pl-VAP) is a member of the copper-containing amine oxidase/semicarbazide-sensitive amine oxidase (AOC/SSAO) enzyme family. It is continuously expressed as a transmembrane glycoprotein in the vascular wall during development and facilitates the accumulation of inflammatory cells into the inflamed environment. It has been shown to be released in cerebral ischemia.

E-selectin (CD62E), is a selectin cell adhesion molecule, expressed only in endothelial cells when activated by cytokines in the setting of inflammation.

Blood levels of syndecan-1, thrombomodulin, pl-VAP, and E-selectin in individuals with TTP were studied. Thrombotic thrombocytopenic purpura (TTP) is a debilitating hematological disorder characterized by recurrent attacks of thrombocytopenia (low platelets) and thrombosis associated with hemolytic anemia. Previous reports suggest that a subset of individuals with TTP develop cognitive impairment in the course of their disease which is thought to be due to chronic endothelial injury in the brain. Such changes are not usually apparent on standard brain imaging techniques as these do not have adequate resolution to demonstrate changes at a cellular level.

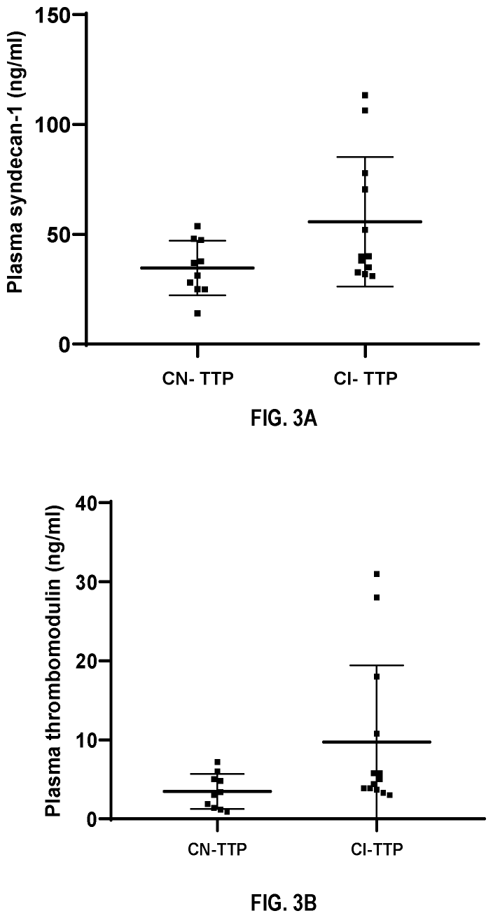

Plasma levels of syndecan-1, thrombomodulin, pl-VAP, and E-selectin were studied in a cohort of individuals with TTP enrolled in a clinical study at OSU. These individuals included those with (n=12) and those without (n=10) evidence of cognitive impairment (based on results of formal neuropsychological testing using the Cogstate). Blood levels were collected between clinical attacks (i.e. during remission) with a median interval of 6 months between blood collection and last clinical relapse. In this cohort, plasma syndecan-1 (55.7 ng/ml, p=0.04) and thrombomodulin (9.7 ng/ml, p=0.05) levels were significantly increased in TTP patients who had evidence of cognitive impairment compared (CI) to cognitively normal individuals with TTP (CN; 34.7 ng/ml and 3.5 ng/ml, respectively) (FIG. 3). There were trends for higher plasma levels of pl-VAP and E-selectin in cognitively impaired, compared to cognitively normal, individuals with TTP that did not reach statistical significance in this small cohort (data not shown). These findings support the concept that cognitive impairment in TTP is due to endothelial injury in the brain. These data also support the concept that endothelial injury in the brain is associated with higher levels of endothelial injury markers in the blood. Therefore, blood levels of these markers have a strong potential to be commercialized into blood tests to detect endothelial damage in the brain.

### Example 4: Autoimmune Neurological Disorders

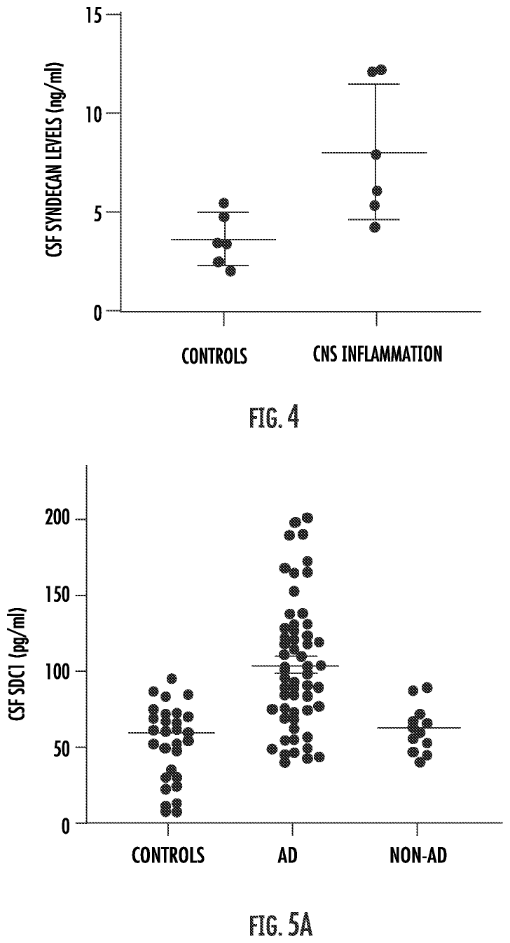

In this study, syndecan-1 levels were measured in CSF samples from cognitively normal healthy controls (n=6) and cognitively normal individuals with multiple sclerosis or CNS autoimmune disease (n=6) which were obtained from the OSU Biobank. CSF syndecan-1 levels were elevated in individuals with MS/autoimmune disease (8.0 ng/ml; p=0.015) compared to controls (3.7 ng/ml) (FIG. 4). These findings suggest that endothelial injury in the setting of MS and autoimmune disorders can be measured using CSF syndecan-1 levels.

### Example 5: Endothelial Markers and Alzheimer's Disease

Within neurodegenerative disorders, data disclosed herein shows that these marker elevations can be relatively specific for AD compared to Parkinson's disease or Frontotemporal dementia. Endothelial dysfunction is a common pathology in AD and other neurological disorders and refers to characteristic structural and functional changes in endothelial cells. In AD, endothelial dysfunction is observed in ˜80% of AD brains, including those that do not show signs of small vessel disease (arteriosclerosis), large vessel disease (atherosclerosis), or amyloid angiopathy. Recent data suggest that endothelial dysfunction is a central pathological substrate which precedes abnormal protein aggregation in AD, and directly contributes to synaptic injury and cognitive impairment. RNA-sequencing (RNA-seq) analyses suggest that endothelial pathways are among the most differentially expressed in human AD brains, including those that do not display signs of cerebrovascular pathology. Consistent with these reports, 30 out of 45 genes associated with AD were found to be expressed in endothelial cells, and several of these AD genes had their highest expression levels in endothelial structures. Therefore, disclosed herein are markers that reflect injury to the brain endothelium in the absence of trauma or microscopic/macroscopic evidence of vessel disease which is expected to affect endothelial cells in addition to other vascular constituents such as smooth muscle, fibroblasts, and connective tissue.

Reliable markers of endothelial injury allow for better understanding the role of this pathology in AD pathogenesis including its contribution to protein aggregation (amyloid and tau) and direct effects on neuronal and synaptic integrity or functions leading to cognitive impairment. The identification of novel markers which can reliably detect and measure endothelial dysfunction in early stages of AD pathogenesis has significant clinical and research implications including:

CSF endothelial markers improve the diagnostic ability of CSF tau, p-tau181 and Aβ342 in preclinical AD when combined with other AD biomarkers. SDC1 and SDC4 also differentiate AD from non-AD dementias to a better extent than Aβ342 and tau: As endothelial injury occurs very early in AD, endothelial markers can complement the diagnostic and prognostic value of established AD biomarkers (CSF p-tau181 and Aβ42) in early symptomatic or pre-symptomatic AD and improve patient selection and stratification for AD treatment or prevention trials.

It is shown herein that all 4 endothelial markers (SDC1, SDC4, VEC, and SELE) are significantly increased in AD compared to healthy controls and non-AD dementias (PD and FTD) and complement the diagnostic utility of established AD biomarkers (tau, p-tau181 and Aβ42) in differentiating AD from non-AD dementias. CSF SDC1 levels were examined in individuals with early symptomatic AD (CDR 0.5-1; n=56, mean age, 78 years; range, 60-88 years), cognitively normal controls (CDR 0; n=29, mean age, 77 years) and non-AD dementias (n=12; including frontotemporal dementia, n=6; and Parkinson's disease; n=6). Participants with vascular risk factors (i.e. hypertension, hyperlipidemia, diabetes mellitus, smoking, and obstructive sleep apnea), traumatic brain injury (TBI), history of cerebrovascular events (ischemic or hemorrhagic strokes), or imaging evidence of small or large vessel disease were excluded.

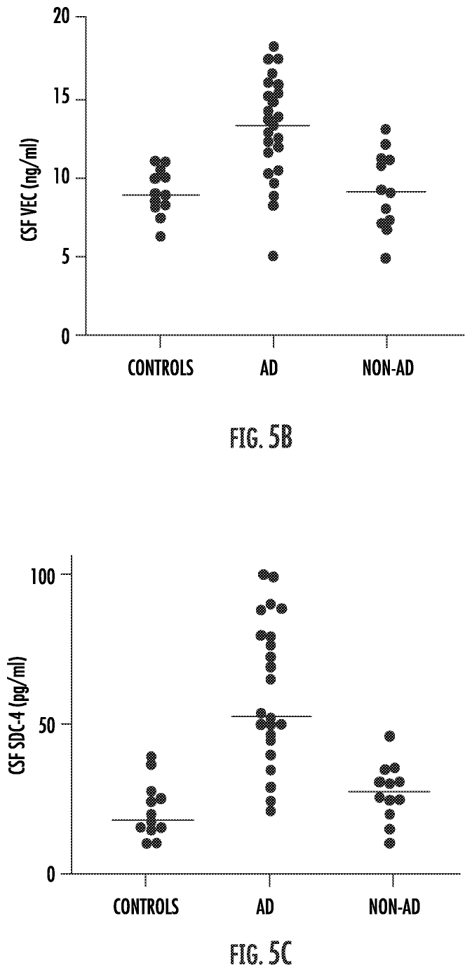

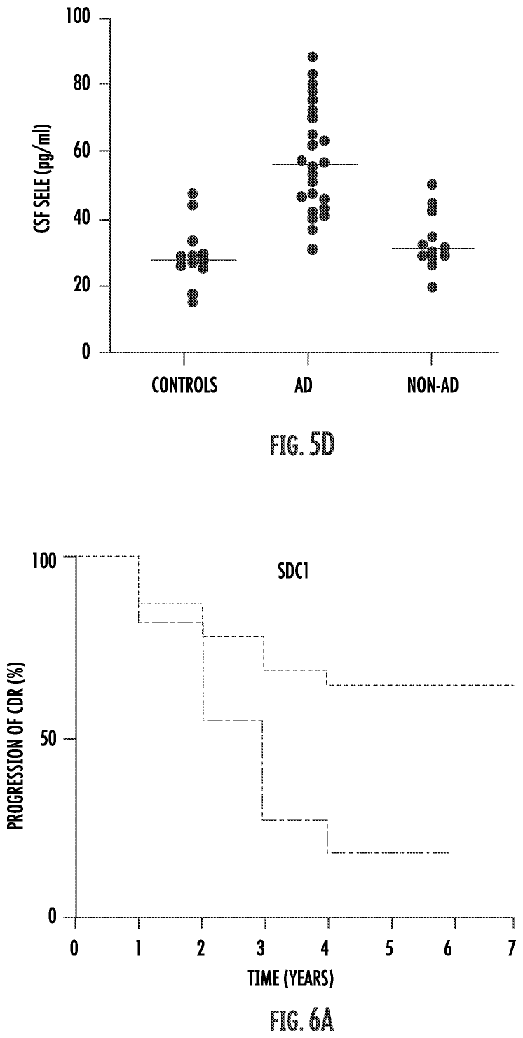

In this cohort, CSF SDC1 levels were significantly elevated in biomarker-confirmed AD (mean±SE, 103.8±6 pg/ml; n=56) compared to healthy controls (52.8±4.6 pg/ml; n=29) and non-AD dementias (61.6±4 pg/ml, n=12, p<0.0001) (FIG. 5A). CSF VEC, SDC4, and SELE levels were examined in a subset of this cohort (n=48). CSF VEC levels were significantly higher in AD (mean±SE; 13.1±0.6 ng/ml; n=24) compared to healthy controls (9.1±0.4 ng/ml; n=12) and non-AD dementias (9.2±0.7 ng/ml; n=12) (p<0.0001) (FIG. 5B). Similarly, CSF SDC4 (61.3±5 pg/ml; n=23) and SELE (57.3±3 pg/ml; n=24) levels were significantly elevated in AD compared to controls (SDC4, 21.5±3 pg/ml; SELE, 28.9±3 pg/ml; n=12) and non-AD dementias (SDC4, 27.4±3 pg/ml; SELE, 32.8±3 pg/ml, n=12) (p<0.0001) (FIG. 5C-D), adjusting for age, gender, and APOE4 genotype. Unadjusted ANOVAs are shown in FIG. 5 for illustration (bars represent mean values).

The diagnostic utility of CSF SDC1, SDC4, VEC, and SELE in AD is comparable to that of classic markers of AD pathology (total tau, p-tau181, and Aβ42) and improves the differentiation between AD and non-AD dementias. Receiver Operating Characteristic (ROC) curves compared the diagnostic utility (i.e. combination of sensitivity and specificity) of CSF SDC1, SDC4, VEC, and SELE to that of established CSF AD biomarkers (CSF total-tau, p-tau181, and Aβ42) in differentiating AD from controls and non-AD dementias. The Area Under the Curve (AUC) for established CSF AD biomarkers in this cohort were 0.93 for p-tau181, 0.91 for Aβ42, and 0.89 for t-tau (p<0.0001). The AUC was 0.87 and 0.84 (p<0.0001) for CSF SDC1, 0.90 and 0.92 (p=0.0001) for CSF SDC4, 0.86 and 0.85 (p=0.0004) for CSF VEC, and 0.89 and 0.93 (p<0.0001) for CSF SELE in differentiating AD from controls, or AD from combined cohorts of controls and non-AD dementias, respectively. In this cohort, CSF SDC4 (0.92, p<0.0001) and SELE (0.94, p<0.0001) levels differentiated AD from non-AD dementias (PD and FTD) to a better extent than CSF t-tau (0.87, p=0.0001) or CSF Aβ42 (0.75, p=0.0004).

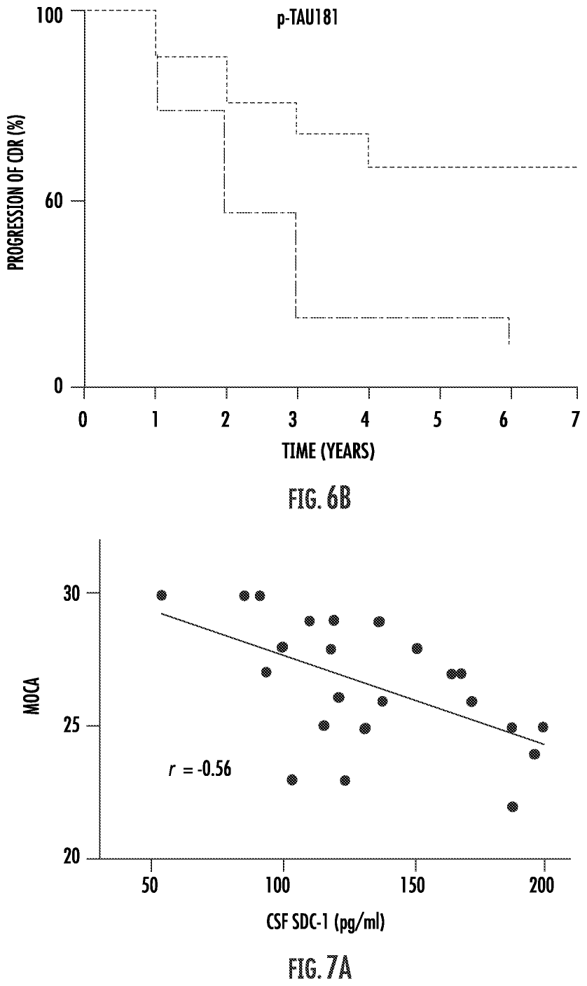

Endothelial markers improve the prognostic utility of established AD biomarkers to predict clinical progression in AD over time, including future cognitive impairment in cognitively normal individuals. Of the n=85 individuals in the AD and control cohorts, a subset (n=35; including n=17 and n=18, respectively, with CDR 0 and CDR >0 at baseline) progressed in their CDR categories over follow-up (1-7 years). In this subset, baseline CSF SDC1 (hazard ratio [HR] 6.7, p=0.01), t-tau (HR 8.3, p=0.004), p-tau181 (HR 7.5, p=0.006), but not Aβ42, levels predicted clinical progression over time (adjusting for age, sex, education, and APOE4) (FIG. 6). Importantly, the addition of CSF SDC1 to CSF p-tau181 and Aβ42 (time-dependent AUC of 0.83, p=0.003) improved their collective predictive ability (AUC 0.71) for clinical progression. Similarly, the combination of CSF SDC1, tau, and Aβ42 (0.88; p=0.001) was a stronger predictor of future cognitive decline than the combination of CSF tau and Aβ42 (0.75). Cox proportional hazard models for the risk of progression across CDR categories over time are shown in FIG. 6. Individuals with SDC1 or p-tau181 levels in the upper tercile of values (shown in red) were more likely to progress over time compared to those with lower values (shown in blue).

Combining the markers disclosed herein with other AD markers significantly improves the ability to detect preclinical AD, differentiate AD from non-AD dementias, and predict the risk of clinical progression over time to a much better extent than what is possible with current AD markers.

In addition to their value as diagnostic and prognostic markers, our data suggest that endothelial markers can measure the contribution of endothelial dysfunction to brain atrophy, neuronal/synaptic injury, and cognitive decline independently of amyloid and tau. Therefore, these markers improve the ability to track and monitor clinical, cognitive, and radiological progression to a better extent than markers of amyloid or tau alone. The relationship between endothelial injury, brain atrophy, and cognition in AD, independently of amyloid and tau (adjusting for age, sex, APOE4, and baseline CSF p-tau181 and Aβ42) was examined.

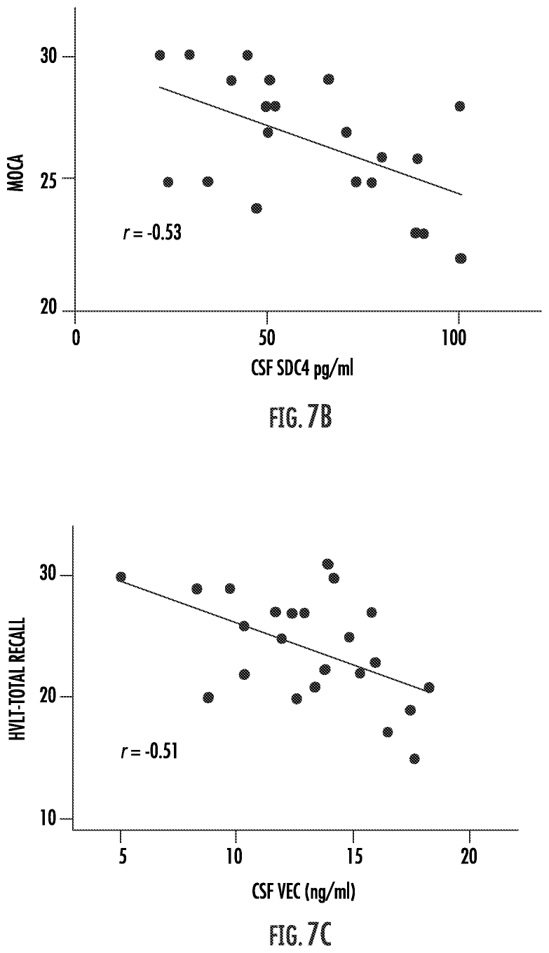

Higher CSF SDC1 (r=−0.56, p=0.005; r=0.64, p=0.002) and CSF SDC4 (r=−0.53, p=0.01; r=0.44, p=0.04) levels were associated with lower global cognitive performance, indicated by lower scores on the Montreal Cognitive Assessment (MOCA) (FIG. 7A-B) and higher scores on the CDR-sum of boxes (CDR-SB), respectively.

CSF VEC demonstrated weaker correlations with global cognition (MOCA, r=−0.44; p=0.05) but was closely associated with episodic memory scores on the Hopkins Verbal Learning Test (HVLT) (r=−0.51, p=0.01) (FIG. 7C), which is consistent with the association of episodic memory deficits with amyloid in early AD.

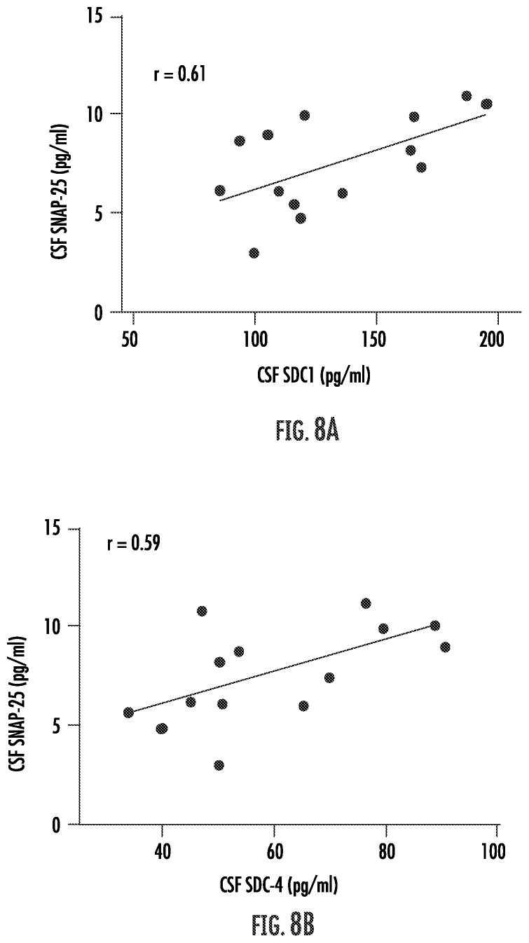

Consistent with these findings, higher CSF SDC1 (r=0.61; p=0.03) and SDC4 (r=0.59, p=0.02) levels were associated with higher CSF SNAP-25 levels reflective of more severe synaptic injury in AD (FIG. 8).

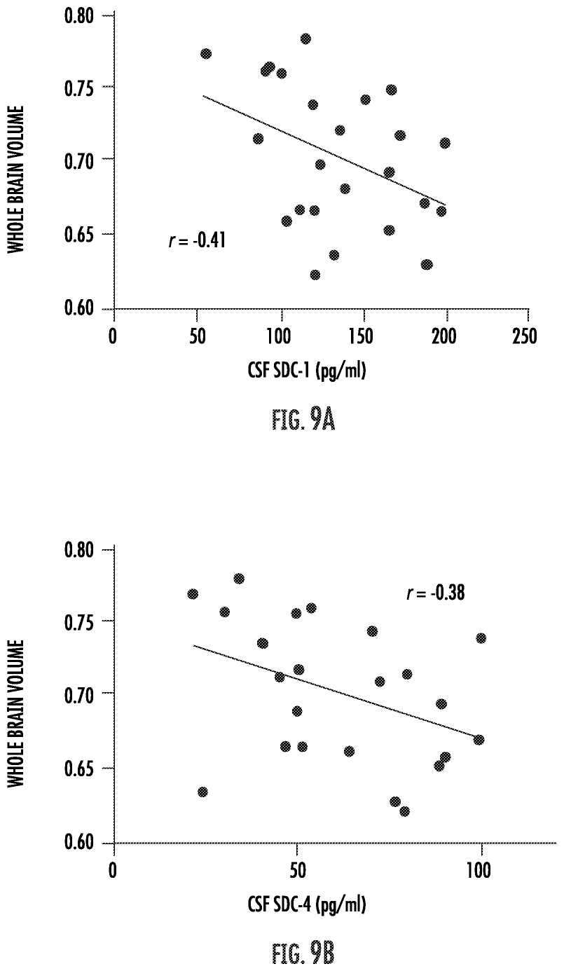

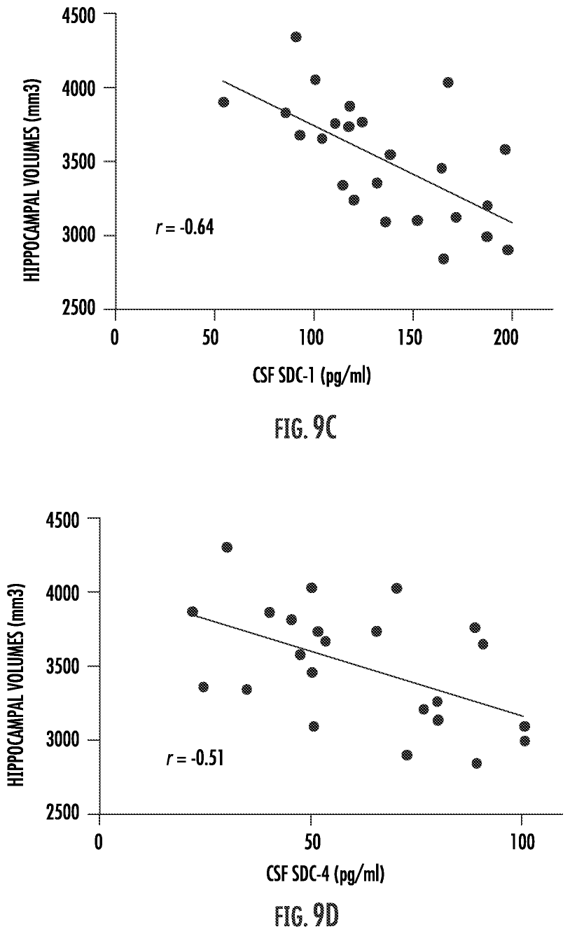

Higher CSF SDC1 levels in the AD cohort correlated with lower whole brain (r=−0.41, p=0.04) and hippocampal (r=−0.64, p=0.001) volumes, reflective of more severe neurodegeneration (adjusting for age, sex, and APOE4). CSF SDC4 levels also correlated with lower whole brain (r=−0.38; p=0.05) and hippocampal (r=−0.51, p=0.01) volumes in AD (FIG. 9).

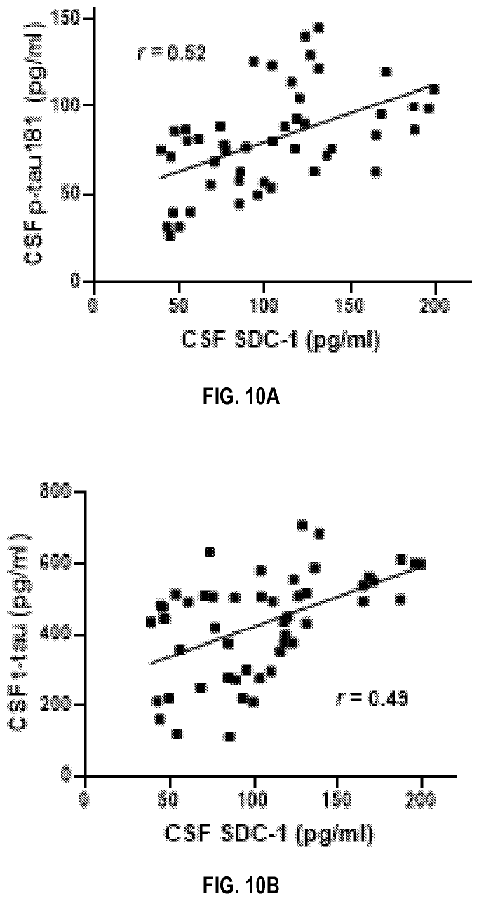

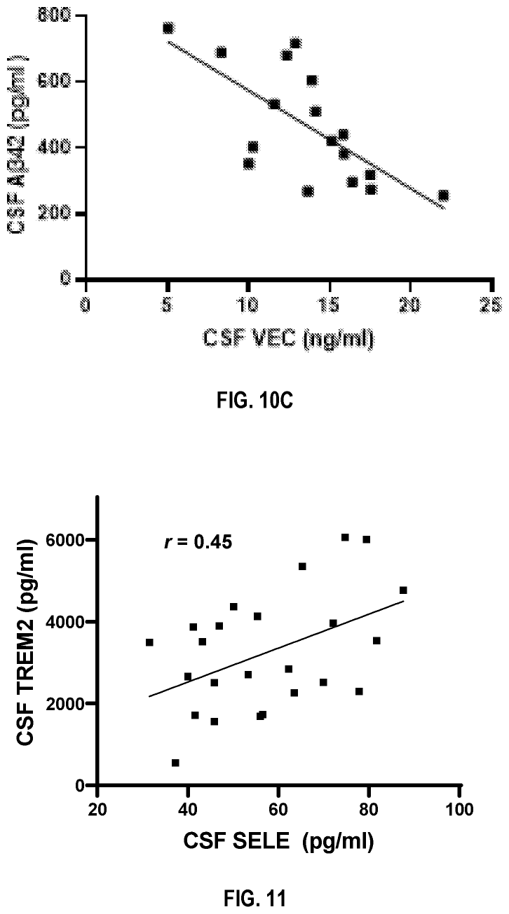

Higher CSF SDC1 levels in the AD cohort correlated with higher CSF p-tau181 (r=0.52, p=0.0002) and CSF tau levels (r=0.49, p=0.0002) which reflect tau pathology (adjusting for age, sex, and APOE4) in the AD cohort. CSF VEC levels were strongly associated with amyloid pathology. Higher CSF VEC levels in the AD cohort correlated with lower CSF Aβ42 (r=−0.53, p=0.03) (adjusting for age and sex) consistent with more severe amyloid pathology. CSF SDC1 also correlated with CSF Aβ42 (r=−0.40; p=0.05) and cortical amyloid burden (total cortical SUVR) on amyloid PET-PM scans (r=0.37; p=0.06) in the AD cohort (FIG. 10). CSF SELE levels correlated with CSF sTREM2 (r=0.45, p=0.02) a surrogate for microglial activation and inflammation. These data suggest that higher CSF SELE levels, reflective of more severe CNS endothelial injury, are associated with microglial dysregulation and inflammation in AD (FIG. 11).

Safety Assessment Tools in Clinical Trials of amyloid-targeting therapies: Endothelial damage is a serious adverse event associated with investigational AD therapies which target vascular amyloid. The anti-amyloid agent aducanumab (Aduhelm from Biogen) has recently become the first FDA-approved disease-modifying treatment for AD and will likely be widely administered in clinical and research settings. There is an urgent need to identify novel markers of endothelial injury to help detect and monitor the earliest signs of amyloid-related imaging abnormalities (ARIAs) associated with this treatment. Endothelial injury markers can provide valuable tools to identify early signs of ARIAs (which likely precede imaging findings) and facilitate proper screening of patients who may be at higher risk for such complications. For example, these markers can be used to identify patients who already have high endothelial injury and therefore would be at risk for these ARIAs. For those who develop ARIAs, these markers will assist in determining whether they are improving or getting worse in response to ARIA treatment (usually with steroids and blood pressure management).

New Knowledge regarding Spatiotemporal Patterns of AD Biomarker Changes and New Models for AD: Current hypothetical disease models and biomarker-based definitions of AD are limited to amyloid, tau, and neurodegeneration. These models are criticized for the fact that they do not include or exclude other pathologies that are commonly observed in AD brains and contribute to neuronal/synaptic injury and cognitive impairment, including endothelial injury. In fact, a recent data-driven model which examined biomarker changes over a decade or so in 1100 individuals with AD and controls that span 5 decades of aging (40-90), suggests that alterations to vascular/blood flow precede amyloid or tau deposition and are the first changes to be observed in AD brains.

These data-driven models highlight the need to incorporate endothelial and other vascular constituents into disease models to better understand the pathological changes that occur in AD brains, and this cannot be achieved without biomarkers. These new endothelial markers disclosed herein fill this gap.

The markers disclosed herein were identified through whole-brain and single-nucleus (sn) RNA-seq analyses of human AD brains: whose brain RNA and CSF protein levels are significantly altered in AD compared to controls. Several characteristics support the utility of these proteins as markers of endothelial dysfunction in AD, including i) abundant expression in brain endothelium, well-established roles in mediating endothelial cell proliferation or architecture, iii) significantly altered brain and CSF expression levels in AD compared to controls or other neurodegenerative disorders, and iv) the feasibility of accurate quantification of their levels in CSF and brain-derived blood exosomes (BE).

In a pilot study, brain snRNA-seq data was analyzed from n=107 participants with neuropathologically-confirmed AD and controls who were ≥65 years and had no prior history of TBI or pathological evidence of cerebrovascular disease. Brain SDC1 (p<0.01), SDC4 (p<0.01), SELE (p<0.0001), and CDH5 (gene for VEC; p<0.0001) were differentially expressed by ≥2-fold change in individuals with higher (2-3) vs lower (0-1) National Institute of Aging (NIA)-Reagan Scores. SDC1, SDC4, and SELE brain RNA levels were increased, while those of VEC were decreased, in AD. These findings suggest that higher CSF VEC levels in AD reflect endothelial cell loss and the release of abundant endothelial proteins into the CSF, whereas increased CSF SDC1, SDC4, and SELE levels reflect both increased release from endothelial and increased brain endothelial expression in AD.

### Example 6: Value of CSF Markers of Endothelial Injury in Other Conditions Other than Ad Including Vascular Injury, and their Influence on Neuronal and Synaptic Integrity and Contribution to Cognitive Impairment

Brain expression levels of SDC1, SDC4, and SELE are increased in AD and reflect disease-specific changes in regulatory expression. Similar changes are not seen with other neurodegenerative disorders in FTD and PD cohorts.

On the other hand, these proteins can also be measured in other conditions to reflect non-specific endothelial injury. This is not novel and elevation of other endothelial proteins in stroke and other vascular disorders has been reported. Such conditions include stroke, trauma, TTP, vasculitis, among other conditions. In these cases, any endothelial protein can increase (just like cardiac enzymes are leaked into the blood with during a myocardial infarction). However, in these cases, the value of measuring these proteins is that they offer a measure of disease severity and monitoring progression or response to treatment over time.

In this regard, disclosed herein is a method of using these markers to determine whether vascular injury can be successfully treated by demonstrating lower levels of endothelial injury markers after treatment compared to before treatment.

### Role of Endothelial Dysfunction in AD Pathogenesis

### Established Mechanisms Implicating Endothelial Dysfunction in AD:

↓Endothelial GLUT1 transporter→impaired neuronal glucose uptake
↓LRP-1, ←MEOX2, and ↑RAGE→↑Aβ aggregation
↓Endothelial BACE expression→↑Aβ production
Endothelial response to hypoxia→↑reactive oxygen species (ROS)
Aβ→↑JNK/NFkB pathways→↑endothelial pro-inflammatory mediators
Inflammatory milieu of AD→↑endothelial antigen-presentation (↑MHCl/II)
↓secretion of endothelial VEGF and TGF-β→impaired angiogenesis

### Novel Mechanisms Implicating Endothelial Dysfunction in AD:

SDCs interact with APOE and ↑Aβ fibrillization
SDCs increase tau and synuclein aggregation via lipid-raft endocytosis
SDCs interact with SEMA6D, neuropilin/plexin-A1 in neuronal repair
SDCs interact with flotillin/SALM4 which regulate synaptic plasticity
SELE interacts with TREM2 which regulates microglial activity

SDCs interact with APOE4, an important mediator of amyloid and tau pathology: Using STRING and Ingenuity Pathway Analyses (IPA), important interactions between SDC1, SDC4, and APOE4 have been identified, the most significant genetic risk factor for AD which has important roles in amyloid and tau aggregation and synaptic injury. SDC1 promotes the uptake of extracellular APOE4-containing lipoproteins, independently of other APOE4 receptors.

SDCs promote Aβ fibrillization and amyloid plaque formation: Aβ binds to the sulfate chains of SDCs on the endothelial surface through the N-terminal HHQK motif and electrostatic interactions which facilitate Aβ fibrillization. Increased endothelial SDC expression also impairs vascular clearance of amyloid. SDCs are found in amyloid plaques and co-localize with AP deposits in human and mouse AD brains. Overexpression of heparanase is associated with reduced amyloid plaque burden due to increased SDC turnover.

SDCs are implicated in lipid-raft endocytosis (LRE) through interactions with flotillin-1: LRE facilitates Aβ synthesis by β-secretase and promotes tau aggregation⁴⁸ as clusters of raft-derived membranes including flotillin-1 are found within neurofibrillary tangles. Novel interactions between SDCs and flotillin-1 have been identified, which is consistent with reports that SDC overexpression is associated with increased intracellular uptake, transcellular spread, and seeding of misfolded tau aggregates.

SELE (CD62) is an inducible endothelium-specific protein which is exclusively expressed in response to endothelial cell activation by cytokines. It has been found that SELE interacts with the microglial toll-like receptor 2 (TLR2), a primary receptor for Aβ on microglia which activates the pro-inflammatory M1-phenotype of microglia in response to Aβ. Increased SELE in AD can contribute to impaired microglial response to Aβ.

Endothelial proteins are directly involved in molecular pathways related to synaptic plasticity, post-synapse organization, and axonal growth or repair. Several novel links between endothelial proteins and pathways involved in neurogenesis, synaptic, and axonal repair have been identified. SDCs modulate the activity of semaphorin6D (SEMA6D) which interacts with neuropilin/plexin-A1 (NRP-1/PLXNA) and Nr-CAM through PLXNA and Nr-CAM are expressed on neuronal surfaces and regulate axonal growth during development or in response to injury. Therefore, SDC1 and SDC4 can act as upstream negative modulators of axonal repair mechanisms.

Taken together, these findings support a mechanistic role for endothelial dysfunction in AD pathogenesis, and show that different endothelial injury markers reflect endothelial dysfunction in different stages of disease progression, and therefore, likely provide complementary information regarding various aspects of AD pathology. VEC levels can be altered in close relation to amyloid, followed by alterations in SDC1 and SDC4 in relation to tau and brain atrophy, while SELE levels correlate with inflammation. Importantly, data from AD cohorts support the notion that endothelial dysfunction is associated with neurodegeneration, cognitive impairment, and brain atrophy, and can influence synaptic/neuronal repair mechanisms, independently of amyloid and tau.

### Example 7: Brain Derived Exosomes in Plasma

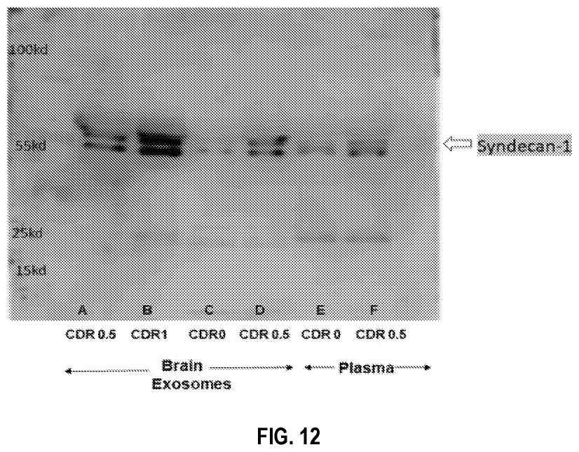

Exosomes derived from the brain endothelium were extracted from plasma samples of individuals with AD and controls for the first time using the exosome extraction protocol described in Abner with modifications [Abner E L, Jicha G A, Shaw L M, Troj anowski J Q, Goetzl E J. Plasma neuronal exosomal levels of Alzheimer's disease biomarkers in normal aging. Annals of clinical and translational neurology. 2016; 3(5):399-403)] and a biotinylated mouse anti-human claudin-5 antibody (Novus Biologicals, Cat. No. NBP2-71329B). Plasma samples from 2 individuals with mild cognitive impairment (MCI) due to AD (Clinical Dementia Rating [CDR] 0.5), one individual with mild AD dementia (CDR 1), and one healthy control (CDR 0) were examined in these studies. Anti-human syndecan-1 antibodies (R&D; DY2780) were used to confirm the presence of syndecan-1 (SDC1) in exosomes derived from human brain-endothelium. As a control group, plasma samples from one participant with MCI due to AD (CDR 0.5) and one healthy control (CDR 0) that did not undergo exosome extraction were also examined for SDC-1 levels. FIG. 12 demonstrates Western blots of these samples. As shown in the figure, SDC1 can be detected in human brain endothelial exosomes (which are extracted from plasma samples) and that brain exosome SDC1 levels are higher in AD (CDR 0.5 or CDR 1) compared to SDC1 brain exosome levels in healthy controls (CDR 0) or plasma SDC1 levels (peripheral; not brain derived).

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

1. A method for detecting endothelial injury in a subject and treating the subject accordingly, the method comprising detecting elevated levels of at least two of the following endothelial markers: syndecan-1, syndecan-4, thrombomodulin, plasmalemmal vesicle-associated protein, E-selectin, and VE-cadherin; and treating the subject accordingly.

2. (canceled)

3. (canceled)

4. (canceled)

5. The method of claim 1, wherein elevated levels are detected for all six of the markers.

6. The method of claim 1, wherein additional biomarker levels are also measured.

7. A method of treating a neurodegenerative disorder in a subject, wherein the method comprises detecting elevated levels of at least two of the following endothelial markers: syndecan-1, syndecan-4, thrombomodulin, pl-VAP, E-selectin, and VE-cadherin; and treating the subject with elevated levels of two or more of these markers with treatments for a neurodegenerative disorder.

8. The method of claim 7, wherein the subject is treated differently based on the results of the presence of at least two of the markers compared to a subject in which the markers were not detected.

9. The method of claim 7, wherein the elevated levels indicate that the subject has Alzheimer's disease.

10. The method of claim 9, wherein the subject has mild cognitive impairment or dementia due to Alzheimer's disease [AD].

11. The method of claim 9, wherein the subject has no symptoms of Alzheimer's disease but has biomarker evidence of AD pathology (preclinical or pre-symptomatic AD).

12. The method of claim 7, wherein the markers are elevated in subjects with Alzheimer's disease but not with those with Parkinson's disease or Frontotemporal dementia.

13. The method of claim 7, wherein the subject is also measured for tau, p-tau181, p-tau217, p-tau231, Aβ42, Aβ38, Aβ42/Aβ38 and Aβ42/Aβ40.

14. The method of claim 7, wherein the endothelial markers predict clinical progression in Alzheimer's disease over time.

15. The method of claim 14, wherein future cognitive impairment in cognitively normal subjects can be predicted.

16. The method of claim 7, wherein the markers are detected in cerebral spinal fluid (CSF).

17. The method of claim 7, wherein the markers are detected in blood.

18. The method of claim 17, wherein the markers are in brain-derived blood exosomes.

19. A method of monitoring effects of a composition on endothelial injury in a subject, the method comprising:
a. measuring levels of at least two of the following endothelial markers in the subject: syndecan-1, syndecan-4, thrombomodulin, plasmalemmal vesicle-associated protein, E-selectin, and VE-cadherin;
b. administering the composition to the subject;
c. again measuring levels of at least two of the following endothelial markers in the subject: syndecan-1, syndecan-4, thrombomodulin, plasmalemmal vesicle-associated protein, E-selectin, and VE-cadherin;
d. determining a significant change in the level of the markers from step a) to step c); and
e. modifying an amount of the composition given, or discontinuing administration of the composition.

20. The method of claim 19, wherein the amount of the composition is decreased or discontinued when a significant increase in at least one of the endothelial markers is found.

21. The method of claim 19, wherein the composition is used to treat vascular injury.

22. The method of claim 21, wherein the amount of the composition is increased or kept the same when one or more markers is significantly decreased.

23. The method of claim 19, wherein the subject has AD.

24. The method of claim 23, wherein the composition being administered is used to treat AD.

25-29. (canceled)

30. A kit comprising antibodies for detecting at least two of the following markers: syndecan-1, syndecan-4, thrombomodulin, plasmalemmal vesicle-associated protein, E-selectin, and VE-cadherin.

31-96. (canceled)
